(19) United States
(12) Patent Application Publication
(10) Pub. No.: US 2024/0109875 A1
(43) Pub. Date: Apr. 4, 2024

(54) NOVEL METHOD FOR SYNTHESIZING NCA COMPOUNDS

(71) Applicants: ECOLE NATIONALE SUPERIEURE DE CHIMIE DE MONTPELLIER (ENSCM), MONTPELLIER CEDEX 5 (FR); UNIVERSITE DE MONTPELLIER (UM), MONTPELLIER (FR); CENTRE NATIONAL DE LA RECHERCHE SCIENTIFIQUE, Paris (FR)

(72) Inventors: Guillaume LACONDE, MONTPELLIER (FR); Jean MARTINEZ, CAUX (FR); Muriel AMBLARD-CAUSSIL, SAINT VINCENT DE BARBEYRARGUES (FR)

(73) Assignees: ECOLE NATIONALE SUPERIEURE DE CHIMIE DE MONTPELLIER (ENSCM), MONTPELLIER (FR); UNIVERSITE DE MONTPELLIER (UM), MONTPELLIER (FR); CENTRE NATIONAL DE LA RECHERCHE SCIENTIFIQUE, Paris (FR)

(21) Appl. No.: 18/259,089

(22) PCT Filed: Dec. 21, 2021
(86) PCT No.: PCT/EP2021/086981

§ 371 Date: Jun. 23, 2023

(30) Foreign Application Priority Data
Dec. 23, 2020 (FR) FR2014040

(51) Int. Cl. C07D 413/06; C07D 263/06; C07D 498/04

(52) U.S. Cl. CPC C07D 413/06; C07D 263/06; C07D 498/04

(57) ABSTRACT

A novel method for synthesizing NCA compounds. Also, a new use of a peptide coupling agent. The method makes it possible to obtain NCA compounds from α-amino-acids, under mild and non-racemic reaction conditions, and in the absence of constraining reagents of use, such as phosgene, which may lead to the formation of undesirable by-products.

The invention relates to a novel method for synthesizing NCA compounds. It also concerns a new use of a peptide coupling agent.

N-carboxyanhydride compounds, or NCAs, are chemical derivatives with high added value industrially used in particular in the production of drugs or polymers. For example, NCAs are intermediates of choice for peptide synthesis under “solvent-free” ecological conditions.

The synthesis of NCA compounds is traditionally carried out with phosgene or a phosgene derivative. However, the use of these reagents is problematic and requires special precautions and facilities, particularly because of their toxicity and dangerousness. In addition, this strategy leads to the concomitant formation of HCl, which is difficult to remove from the NCA product, especially during a large-scale process. However, the presence of residual HCl in the NCA product may give rise to parasitic reactions during its use, for example in a polymerization process.

Some alternative syntheses exist, making it possible to avoid phosgene. Among these methods is the use of carbonyldiimidazole, or diphenyl carbonate. These methods are not industrially applicable, as the presence of secondary products, imidazole and phenol respectively, complicates the purification of the synthesized NCA product. Another method, involving a nitrosation reaction, leads to the concomitant formation of nitric oxide, a toxic compound.

There is therefore a real need for alternatives for the preparation of NCAs compounds, in particular environmentally friendly processes, and to obtain products with a good yield and excellent chemical purity.

One of the objects of the invention is to use the propylphosphonic anhydride reagent in the preparation of NCA compounds.

One of the objects of the invention is to provide a new method for synthesizing NCA compounds, without the concomitant formation of HCl.

Another object of the invention is to provide a method for synthesizing NCA compounds, in the absence of phosgene, or one of its derivatives.

One of the objects of the invention is to provide NCA compounds with good chemical and stereochemical purity.

Another object of the invention is to provide new NCA compounds.

Another object of the invention is to be able to use NCA compounds thanks to their improved chemical purity, in particular as synthetic intermediates in the preparation of a polymer or a UNCA compound.

A first object of the present invention is the use of propane-phosphonic acid anhydride for the preparation of a NCA compound, from a α-amino acid compound N-protected on the α amine function by a linear or branched C₁ to C₂₀—C(O)—O-alkyl substituent, in particular by a tert-butyloxycarbonyl group.

The inventors surprisingly found that α-amino acids can be transformed into NCA compounds, by the action of propylphosphonic anhydride. This transformation thus makes it possible to obtain said NCA compounds with good yields, and excellent chemical and stereoisomeric purities.

The transformation is all the more surprising since other agents conventionally used in peptide chemistry, such as DCC, BOP or HATU, do not lead, or very little, to the formation of NCAs.

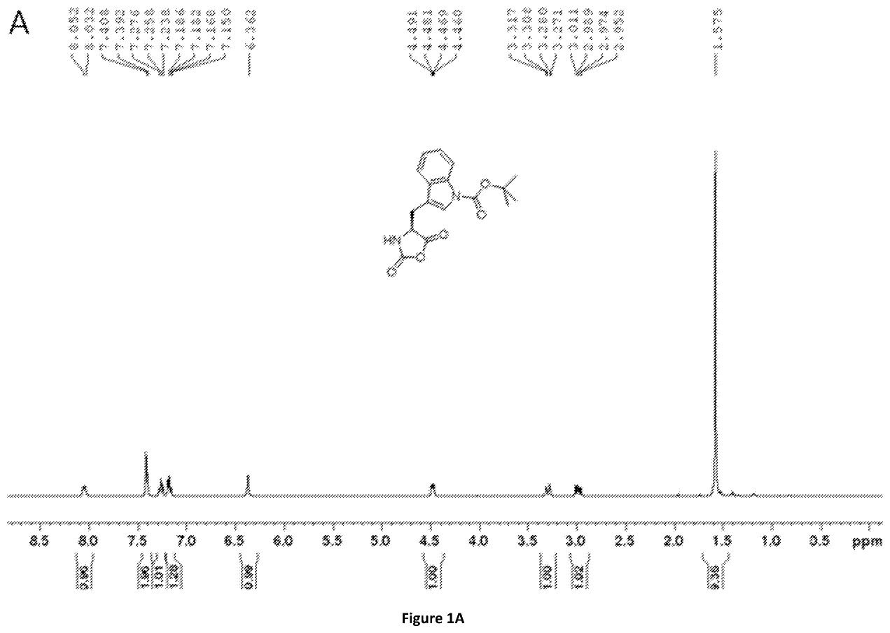

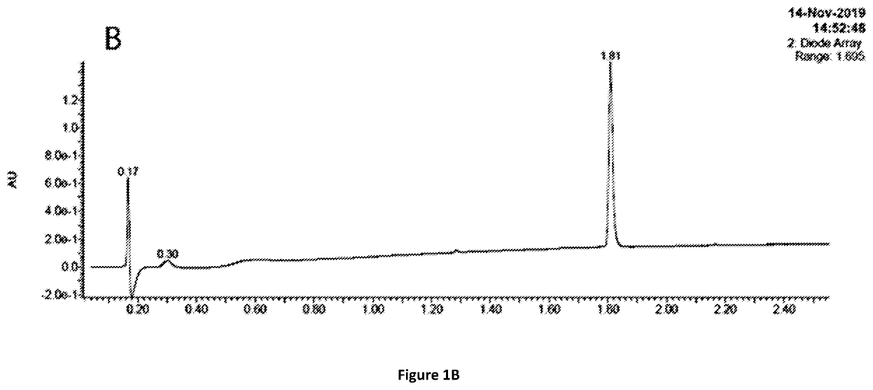

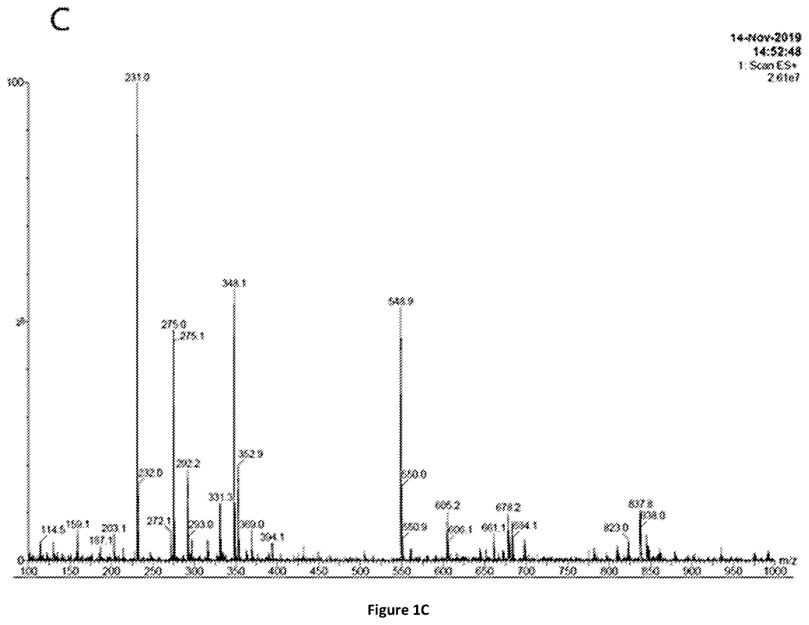

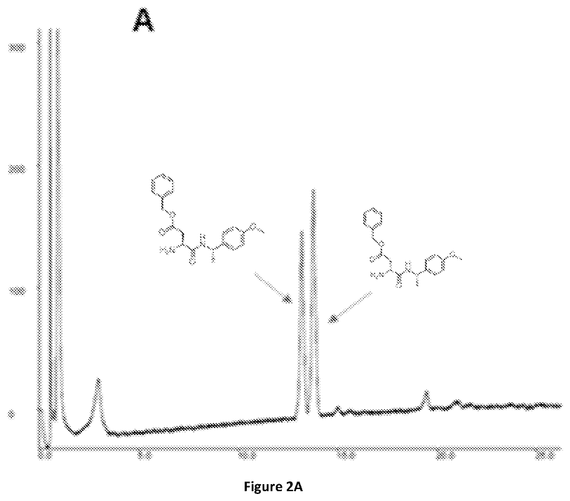

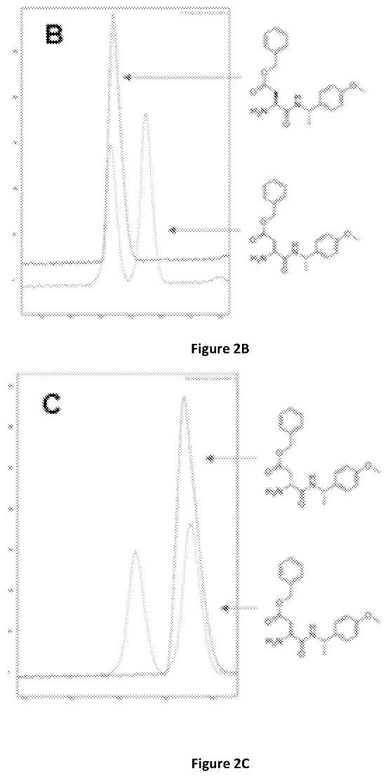

FIGS. 1 and 2, attest to the excellent purity with which the NCA compounds according to the invention are obtained.

By “propane-phosphonic acid anhydride” means a molecule whose structure is shown below. It is an anhydride of propylphosphonic acid, which is in the form of a cyclic trimer on which propyl groups are bonded to phosphorus atoms.

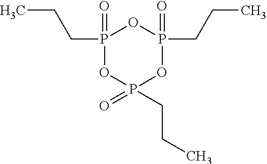 [SMILES: CCCP1(=O)OP(=O)(CCC)OP(=O)(CCC)O1]

### Chemical Structure of Propane-Phosphonic Acid Anhydride

### IUPAC: 2,4,6-Tripropyl-1,3,5,2λ⁵,4λ⁵,6λ⁵-trioxatriphosphinane-2,4,6-trione

The reagent is commercially available, under the name T₃P®, in solution at 50 w/w % in several solvents, such as ethyl acetate, dimethylformamide, toluene, or tetrahydrofuran.

By “linear C₁ to C₂₀ alkyl” means an alkyl group comprising from 1 to 20 carbon atoms, selected from: methyl C₁, ethyl C₂, propyl C₃, butyl C₄, pentyl C₅, hexyl C₆, heptyl C₇, octyl C₈, nonyl C₉, decyl C₁₀, decyl C₁₁, dodecyl C₁₂, tridecyl C₁₃, tetradecyl C₁₄, pentadecyl C₁₅, hexadecyl C₁₆, heptadencyle C₁₇, octadecyl C₁₈, nonadecyl C₁₉, eicosyl C₂₀, in particular a linear C₁ to C₁₀ alkyl group, in particular a linear C₁ to C₅ alkyl group.

By “branched alkyl” is meant a linear alkyl group as defined above comprising substituents selected from the linear alkyl groups defined above, said linear alkyl groups being also capable of branching. Among the branched alkyl groups include an iso-propyl, sec-butyl, iso-butyl, tert-butyl, sec-pentyl, iso-pentyl, iso-hexyl, iso-heptyl, iso-octyl, iso-nonyl and iso-decyl.

The compounds “NCAs”, or N-CarboxyAnhydrides of α-amino acids, have compounds comprising the unit shown below. These compounds may carry one or more substituents on the carbon α the carbonyl function, as well as on the nitrogen atom.

In the context of the present invention, these NCA compounds are prepared from a N-protected-α-amino acid compound comprising a unit shown below, and wherein R represents an alkyl group C₁ to C₂₀, linear or branched.

The group R is preferably a tert-butyl group, giving rise to a N-protected α-amino-acid the α amine function of which is protected by a Boc group.

In the case where the NCA compound carries a substituent other than a hydrogen atom on the nitrogen, this substituent is also present on the corresponding α-amino-acid molecules.

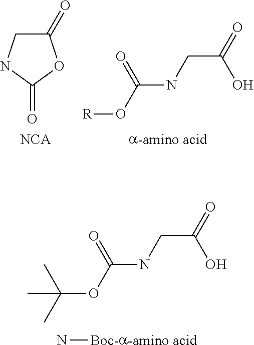

The carboxylic acid function present in the aforementioned N-protected α-amino-acid compound is optionally in the form of carboxylate, especially in the form of a sodium salt, a potassium salt, or a lithium salt.

A specific class of NCA compounds are UNCAs, or N-Urethane-CarboxyAnhydrides of α-amino acids. These compounds comprise, on the nitrogen atom of the NCA ring, a urethane group. UNCA compounds, like other NCAs compounds, may include additional substituents on the carbon of the NCA ring.

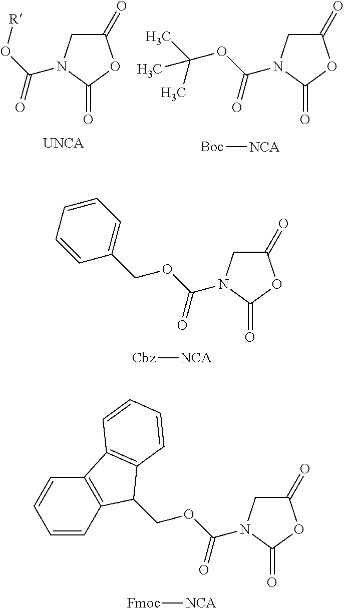

The present invention also makes it possible to obtain this specific class of NCA compounds. In this case, it is advisable to use a double-N-protected α-amino acid on the amine function in α, in the form of dicarbamate. Thus, a N-(Boc)₂₋α-amino-acid, N-(Boc)(Cbz) or N-(Boc)(Fmoc) can for example be used.

According to a particular embodiment, the present invention relates to a use as defined above, wherein the preparation of the NCA compound is made:
either in the presence of an organic base, at room temperature, or
or in the absence of an organic base, at a temperature between 40 and 80° C.

The preparation of a compound NCA, from a N-protected α-amino-acid compound, according to the present invention is preferably carried out in the presence of an organic base, said base being able to promote the reaction. However, the reaction also takes place in the absence of an organic base, but it is slower. In this case, it would be necessary to heat the reaction medium, in order to increase the kinetics of the reaction, and to obtain reaction times compatible with an industrial process.

In the case of the presence of an organic base, the reaction takes place at “room temperature”, i.e. at a temperature between 20 and 30° C., in particular about 25° C.

According to another particular embodiment, the present invention relates to a use as defined above, wherein the organic base is a nitrogenous base, in particular selected from triethylamine, 1,8-diazabicyclo [5.4.0] undec-7-ene, diisopropylethylamine, N-dimethylaminopyridine, N-methylmorpholine or pyridine, preferably pyridine.

According to another particular embodiment, the present invention relates to a use as defined above, wherein the preparation of the compound NCA is done:
either in the presence of an organic base, at room temperature, or said organic base being selected in particular from triethylamine, 1,8-diazabicyclo [5.4.0] undec-7-ene, diisopropylethylamine, N-dimethylaminopyridine, N-methylmorpholine or pyridine,
or in the absence of an organic base, at a temperature between 40 and 80° C.

According to another particular embodiment, the present invention relates to a use as defined above, wherein the preparation of the NCA compound is done in the presence of an organic solvent.

Among the organic solvents used in the preparation of a compound NCA, according to the present invention, mention may be made, but not limited to, ethyl acetate, butyl acetate, tetrahydrofuran, 2-methyltetrahydrofuran, toluene, N, N-dimethylformamide, chlorobenzene, methylene chloride or acetonitrile.

The organic solvent is in particular selected according to the commercial availability of the propane-phosphonic acid anhydride reagent, which is in particular, marketed in 50% solution in ethyl acetate, or dimethylformamide.

According to another particular embodiment, the present invention therefore relates to a use as defined above, wherein the organic solvent is selected from ethyl acetate or dimethylformamide, in particular ethyl acetate.

According to another particular embodiment, the present invention relates to a use as defined above, wherein the propane-phosphonic acid anhydride is used in an amount of 1 to 4 molar equivalents relative to the N-protected α-amino-acid compound.

The amount of propane-phosphonic acid anhydride is in particular 1, 2, or 3 equivalents, in the presence of an organic base, and in particular 2, 3 or 4 equivalents in the absence of an organic base.

According to another particular embodiment, the present invention relates to a use as defined above, wherein the preparation of the NCA compound is made in the presence of an organic base in an amount of 0.25 to 3 molar equivalents relative to the N-protected α-amino-acid compound, in particular 1 to 3 molar equivalents, preferably 3 molar equivalents.

The term “from 0.25 to 3 molar equivalents” also means the following ranges: from 0.25 to 2, from 0.25 to 1, from 0.25 to 0.5, from 0.5 to 3, from 1 to 2, or from 0.5 to 2.

According to another particular embodiment, the present invention relates to a use as defined above, wherein:
the preparation of the compound CNA is made in the presence of an organic solvent, in particular selected from ethyl acetate or dimethylformamide, and/or
the preparation of the NCA compound is done in the presence of an organic base in an amount of 0.25 to 3 molar equivalents relative to the N-protected α-amino-acid compound, and/or
Propane-phosphonic acid anhydride is used in an amount of 1 to 4 molar equivalents relative to the N-protected α-amino-acid compound.

An amount of less than 1 base equivalent, compared to the N-protected α-amino-acid compound, may be used. However, a stoichiometric quantity, or greater than the stoichiometric value, of organic base makes it possible to increase the kinetics of the reaction, in order to be able to reach reaction times compatible with an industrial process.

According to another particular embodiment, the present invention relates to a use as defined above, wherein the NCA compound is of Formula 1:

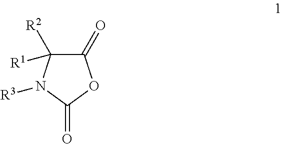

where:
R¹ and R² independently represent:
   a hydrogen atom,
   a group chosen from:
      C₁ to C₂₀ linear or branched alkyl,
      C₂ to C₂₀, linear or branched alkenyl,
      C₃ to C₁₀ cycloalkyl,
      C₃ to C₁₀ heterocycloalkyl, wherein the heteroatom is selected from N, O, and S,
      aryle,
      C₁ to C₂₀ alkyl-aryl, in particular benzyl,
      heteroaryl, wherein the heteroatom is selected from N, O, and S, in particular 3-methylindole,
      C₁ to C₂₀ alkyl-heteroaryl, wherein the heteroatom is selected from N, O, and S,
      a halogen, in particular a fluorine atom,
   said alkyl, alkyl-aryl or alkyl-heteroaryl may be substituted on at least one carbon of the alkyl radical by one or more groups selected from:
      O—R⁴, wherein R⁴ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl, C₁ to C₂₀ alkyl-heteroaryl and a protective group, in particular selected from tBDMS, t-butyl, benzyl, trityl and xanthyl,
      O—C(O)—R⁵, wherein R⁵ is selected from H, linear or branched C₁ to C₂₀ alkyl, linear or branched C₁ to C₂₀ alkyl heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      C(O)—R⁶, wherein R⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      C(O)—O—R⁷, wherein R⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      NR⁸R⁹, wherein R⁸ and R⁹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      (NH)CNHR¹⁰, wherein R¹⁰ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, C₁ to C₂₀ aryl, C₁ to C₂₀ alkyl-aryl, heteroaryl and C₁ to C₂₀ alkyl-heteroaryl and a protective group, in particular NO₂, Pbf, Pmc, Mtr or Boc,
      NR¹¹C(O)R¹², wherein R¹¹ and R¹² are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical-C(O)R¹² being in particular a protective group such as Boc, Cbz, Alloc or Fmoc,
      C(O)—NR¹³R¹⁴, wherein R¹³ and R¹⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      S—R¹⁵, wherein R¹⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
      a halogen, in particular selected from F, Cl, Br and I,
   said aryl, alkyl-aryl, heteroaryl and alkyl-heteroaryl may be substituted on the aromatic or heteroaromatic ring by one or more groups selected from:
      C₁ to C₂₀ linear or branched alkyl,
      C₃ to C₁₀ cycloalkyl,
      C₃ to C₁₀ heterocycloalkyl,
      O—R¹⁶, wherein R¹⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
      O—C(O)—R¹⁷, wherein R¹⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      C(O)—R¹⁸, wherein R¹⁸ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      C(O)—O—R¹⁹, wherein R¹⁹ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, C₁ to C₂₀ aryl, C₁ to C₂₀ alkyl-aryl, C₁ to C₂₀ heteroaryl and C₁ to C₂₀ alkyl-heteroaryl,
      NR²⁰R²¹, wherein R²⁰ and R²¹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      (NH) CNHR²², wherein R²² is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
      NR²³C(O)R²⁴, wherein R²³ and R²⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀-alkyl-heteroaryl, linear or branched C₁ to C₂₀ O-alkyl, linear or branched C₁ to C₂₀ O-alkyl-aryl, the radical —C(O)R²⁴ being in particular a protective group such as Boc, Cbz, Alloc or Fmoc,
      C(O)—NR²⁵R²⁶, wherein R²⁵ and R²⁶ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀alkyl-heteroaryl,
      S—R²⁷, wherein R²⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
      a halogen, in particular selected from F, Cl, Br and I,
   R¹ and R² may form a cycle,
R³ represents:
   a hydrogen atom,
   a linear or branched C₁ to C₂₀ alkyl,
      said group R³ may form a cycle with R¹ or R²,
      said compound of Formula 1 may be in the form of a solvate or a hydrate,
      said groups NR⁸R⁹, (NH) CNHR¹⁰, NR²⁰R²¹ and (NH) CNHR²², heteroaryl, alkyl-heteroaryl and/or heterocycloalkyl may be in a salified form,
      when the compound of Formula 1 comprises a carbon atom, said carbon atom may be ¹³C,
      when the compound of Formula 1 comprises a fluorine atom, said fluorine atom may be ¹⁸F,
      when the compound of Formula 1 comprises a hydrogen atom, said hydrogen atom may be deuterium,
      the asymmetric centers of said Formula 1 compound are of R or S configuration, or a mixture thereof.

By group “linear C₂ to C₂₀ alkenyl” means: a linear alkyl chain comprising from two to 20 carbon atoms, comprise one or more double (s) carbon bond (s). In particular, a linear alkyl chain of 3 to 15 carbon atoms, 3 to 10 carbon atoms, 5 to 20 carbon atoms, 10 to 20 carbon atoms, or 5 to 15 carbon atoms.

By group “branched alkenyl” is meant an alkenyl group as defined above, comprising substituents selected from the list of linear alkyl groups defined above, said linear alkyl groups being also likely to be branched.

By group “C₃ to C₁₀ cycloalkyl” means a cycloalkyl group comprising from 3 to 10 carbon atoms, selected from: cyclopropyl C₃, cyclobutyl C₄, cyclopentyl C₅, cyclohexyl C₆, cycloheptyl C₇, cyclooctyl C₈, cyclononyl C₉, or cyclodecyl C₁₀.

By group “C₃ to C₁₀ hetero cycloalkyl” means a cycloalkyl group comprising from 3 to 10 carbon atoms, and further comprising one or more heteroatoms in the ring, in particular 1 or 2 heteroatom (s).

The term “aryl” refers to an aromatic group comprising 5 to 16 carbon atoms within the aromatic ring, in particular from 6 to 12 carbon atoms, in particular comprising 5, 6, 7, 8, 9, 10, 11, 12, 13, 14, 15 or 16 carbon atoms. The aryl groups according to the present invention may also be substituted, in particular by one or more substituents selected from: a linear or branched C₁ to C₁₀ alkyl group, a linear or branched C₁ to C₁₀ O-alkyl group.

Phenyl, anisyl and naphthyl, o-tolyl, m-tolyl, p-tolyl, o-xylyl, m-xylyl, p-xylyl, are examples of aryl groups according to the present invention.

The term “heteroaryl” refers to an aryl group as defined above, comprising atoms other than carbon atoms, in particular N, O or S within the aromatic ring.

Pyridyl, imidazoyl, indolyl, or furanyl are examples of heteroaryl groups according to the present invention.

Within the meaning of the present invention, the expression “R¹ and R² may form a ring” refers to spirocyclic compounds, preferably from 3 to 10 carbon atoms, as follows, for the general formula 3, wherein R³ is as defined above.

The NCA compounds of Formulas 4, 5 and 6 are specific examples of spirocyclic compounds according to the present invention, comprising respectively a cyclopropyl, cyclopentyl, or cyclohexyl ring.

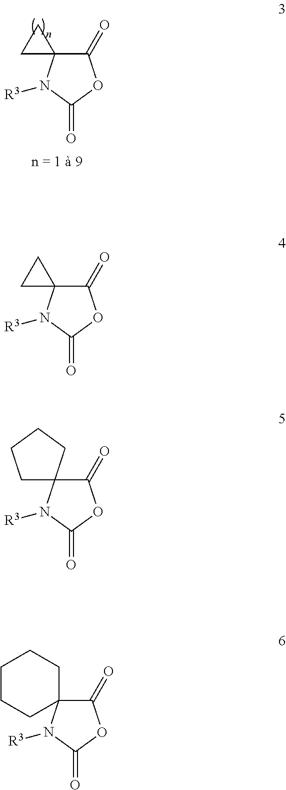

The expression “said group R³ may form a ring with R¹ or R²” refers to the formation of a polycyclic compound, illustrated, non-limitingly, by the following NCA compounds 7 to 9:

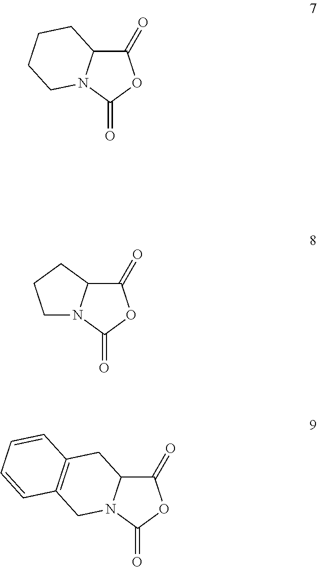 [SMILES: O=C1OC(=O)N2CCCCC12 ; O=C1OC(=O)N2CCCC12 ; O=C1OC(=O)N2Cc3ccccc3CC12]

When the NCA compound comprises an isotope ¹³C, or several isotopes ¹³C, said isotope is either on one of the carbonyl functions of the NCA ring, or on the side chain, represented by R¹ and/or R² in Formula 1. The isotope ¹³C is preferably present as ¹³C═O.

When the NCA compound comprises a deuterium isotope, D, or several D isotopes, said isotope (s) are preferably on a non-enolisable position of the NCA cycle, or on a non-exchangeable position of the side chain, represented by R¹ and/or R² in Formula 1.

The expression “the asymmetric centers of said compound of Formula 1 are of R or S configuration, or a mixture of these configurations” refers to the asymmetric centers formed by the configuration of the groups R¹ and R² (Formulas 10 and 11 below), but also to those possibly present on said groups R¹ and R². This situation is illustrated by Formula 12 below, wherein the group R¹ is a hydrogen atom, and the group R² has a racemic asymmetric center. In this example, the carbon bearing the groups R¹ and R² is of S configuration.

The stereochemistry of NCA molecules is determined by the stereochemistry of the N-protected α-amino-acid from which the NCA compound is formed.

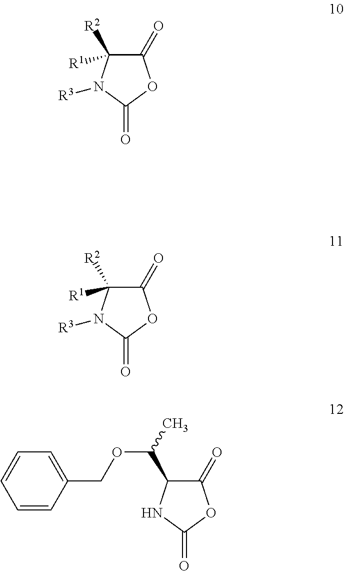

The compound of Formula 1 is preferably diastereoisomerically pure, the excess diastereoisomeric being greater than 80%.

The term “greater than 80%”, greater than 90%, greater than 95%, greater than 98% and in particular greater than 99%.

The compound of Formula 1 is preferably enantiomerically pure, the enantiomeric excess being greater than 80%, the N-protected a amino-acid being in particular of L configuration.

The term “greater than 80%”, greater than 90%, greater than 95%, greater than 98% and in particular greater than 99%.

According to another particular embodiment, the present invention relates to a use as defined above, wherein the compound of Formula 1 is such that:
R¹ and R² independently represent:
   a hydrogen atom,
   a group chosen from:
      a linear or branched C₁ to C₂₀ alkyl,
      a C₃ to C₁₀ cycloalkyl,
      aryle,
      C₁ to C₂₀ alkylaryl, in particular benzyl,
      heteroaryl, wherein the heteroatom is selected from N, O, and S, in particular 3-methylindole,
      C₁ to C₂₀ alkyl-heteroaryl, wherein the heteroatom is selected from N, O, and S,
      a fluorine atom,
   said alkyl, alkyl-aryl or alkyl-heteroaryl may be substituted on at least one carbon of the alkyl radical by one or more groups selected from:
      O—R⁴, wherein R⁴ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
      NR⁸R⁹, wherein R⁸ and R⁹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      (NH)CNHR¹⁰, wherein R¹⁰ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
      NR¹¹C(O)R¹², wherein R¹¹ and R¹² are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R¹² being in particular a protective group such as Boc, Cbz, Alloc or Fmoc,
      S—R¹⁵, wherein R¹⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
   said aryl, alkyl-aryl, heteroaryl and alkyl-heteroaryl may be substituted on the aromatic or heteroaromatic ring by one or more groups selected from:
      alkyl, C₁ to C₂₀, linear or branched,
         O—R¹⁶, wherein R¹⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
         NR²⁰R²¹, wherein R²⁰ and R²¹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
         (NH)CNHR²², wherein R²² is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
         NR²³C(O)R²⁴, wherein R²³ and R²⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R²⁴ being in particular a protective group such as Boc, Cbz, Alloc or Fmoc,
         S—R²⁷, wherein R²⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
         a halogen, in particular selected from F, Cl, Br and I,
   R¹ and R² may form a cycle,
R³ represents:
   a hydrogen atom,
   a linear or branched C₁ to C₂₀ alkyl group,
      said group R³ may form a cycle with R¹ or R²,
      said compound of Formula 1 may be in the form of a solvate or a hydrate,
      said groups NR⁸R⁹, (NH)CNHR¹⁰, NR²⁰R²¹ and (NH)CNHR²², heteroaryl, alkyl-heteroaryl and/or heterocycloalkyl may be in a salified form,
      when the compound of Formula 1 comprises a carbon atom, said carbon atom may be ¹³C,
      when the compound of Formula 1 comprises a fluorine atom, said fluorine atom may be ¹⁸F,
      when the compound of Formula 1 comprises a hydrogen atom, said hydrogen atom may be deuterium,
      the asymmetric centers of said Formula 1 compound are of R or S configuration, or a mixture thereof.

According to another particular embodiment, the present invention relates to a use as defined above, wherein the compound of Formula 1 is such that:
R¹ and R² independently represent:
   a hydrogen atom,
   a group chosen from:
      a linear or branched C₁ to C₂₀ alkyl,
      C₁ to C₂₀ alkyl-aryl, in particular benzyl,
      heteroaryl, wherein the heteroatom is selected from N, O, and S, in particular 3-methylindole,
   said alkyl or alkyl-aryl may be substituted on at least one carbon of the alkyl radical by one or more groups selected from:
      O—R⁴, wherein R⁴ is selected from H, linear or branched C₁ to C₂₀ alkyl, and a protective group, in particular selected from tBDMS, t-butyl, benzyl, trityl and xanthyl,
      NR⁸R⁹, wherein R⁸ and R⁹ are independently selected from H, linear or branched C₁ to C₂₀ alkyl,
      (NH)CNHR¹⁰, wherein R¹⁰ is selected from H, linear or branched C₁ to C₂₀ alkyl, and a protective group, in particular Pbf, Pmc, Mtr or Boc,
      NR¹¹C(O)R¹², wherein R¹¹ and R¹² are independently selected from H, linear or branched C₁ to C₂₀ alkyl, linear or branched C₁ to C₂₀ O-alkyl, linear or branched C₁ to C₂₀ O-alkyl-aryl, the radical —C(O)R¹² being in particular a protective group such as Boc, Cbz, Alloc or Fmoc,
      S—R¹⁵, wherein R¹⁵ is selected from H, linear or branched C₁ to C₂₀ alkyl, and a protective group, in particular trityl or acetamidomethyl (Acm),
   said alkyl-aryl or heteroaryl may be substituted on the aromatic or heteroaromatic ring by one or more groups selected from:
      a linear or branched C₁ to C₂₀ alkyl,
      O—R¹⁶, wherein R¹⁶ is selected from H, linear or branched C₁ to C₂₀ alkyl, and a protective group, in particular selected from tBDMS, t-butyl, benzyl, trityl and xanthyl,
      NR²⁰R²¹, wherein R²⁰ and R²¹ are independently selected from H, linear or branched C₁ to C₂₀ alkyl,
      (NH)CNHR²², wherein R²² is selected from H, linear or branched C₁ to C₂₀ alkyl, and a protective group, in particular NO₂, Pbf, Pmc, Mtr or Boc,
      NR²³C(O)R²⁴, wherein R²³ and R²⁴ are independently selected from H, linear or branched C₁ to C₂₀ alkyl, linear or branched C₁ to C₂₀ O-alkyl, linear or branched C₁ to C₂₀ O-alkyl-aryl, the radical-C(O)R²⁴ being in particular a protective group such as Boc, Cbz, Alloc or Fmoc,
      S—R²⁷, wherein R²⁷ is selected from H, linear or branched C₁ to C₂₀ alkyl, and a protective group, in particular trityl or acetamidomethyl (Acm),
      a fluorine atom,
R³ represents:
   a hydrogen atom,
   a linear or branched C₁ to C₂₀ alkyl group,
      said group R³ may form a cycle with R¹ or R²,
      said compound of Formula 1 may be in the form of a solvate or a hydrate,
      said groups NR⁸R⁹, (NH) CNHR¹⁰, NR²⁰R²¹ and (NH)CNHR²² and/or heteroaryl, which may be in a salified form,
      the asymmetric centers of said Formula 1 compound are of R or S configuration, or a mixture thereof.

According to another particular embodiment, the present invention relates to a use as defined above, wherein the compound of Formula 1 is such that:
R¹ and R² independently represent:
   a hydrogen atom,
   a group chosen from:
      a linear or branched C₁ to C₁₀ alkyl,
      3-methylindole,
   said alkyl may be substituted by one or more groups selected from:
      O—R⁴, wherein R⁴ is a t-butyl group,
      (NH)CNHR¹⁰, wherein R¹⁰ is selected from a protective group, in particular NO₂, Pbf, Pmc, Mtr or Boc,
      NR¹¹C(O)R¹², wherein R¹¹ is H, and the radical —C(O)R¹² is a protective group such as Boc, Cbz, Alloc or Fmoc,
      S—R¹⁵, wherein R¹⁵ is trityl or acetamidomethyl (Acm),
R³ represents:
   a hydrogen atom,
   a linear or branched C₁ to C₂₀ alkyl group,
      said group R³ may form a cycle with R¹ or R²,
      said compound of Formula 1 may be in the form of a solvate or a hydrate,
      said groups NR⁸R⁹, (NH)CNHR¹⁰, NR²⁰R²¹ and (NH)—CNHR²² and/or 3-methylindole may be in a salified form,
      the asymmetric centers of said Formula 1 compound are of R or S configuration, or a mixture thereof.

According to another particular embodiment, the present invention relates to a use as defined above, wherein the compound of Formula 1 is such that R³ is a hydrogen atom.

According to this particular embodiment, the compound of Formula 1 has the structure of Formula 13:

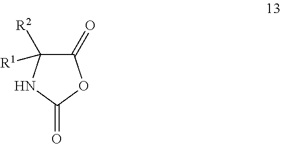

wherein R¹ and R² are as defined above.

According to another particular embodiment, the present invention relates to a use as defined above, wherein the compound of Formula 1 is such that R¹ and R² are identical, and are in particular a methyl.

According to another particular embodiment, the present invention relates to a use as defined above, wherein the compound of Formula 1 is such that R¹ and R² form a cycle.

This embodiment has been illustrated above by the NCA compounds of Formulas 3 to 6.

According to another particular embodiment, the present invention relates to a use as defined above, in which the compound of Formula 1 is such that one of R¹ or R² is a hydrogen atom.

According to this particular embodiment, the compound of Formula 1 has the structure of Formula 14, or Formula 14′:

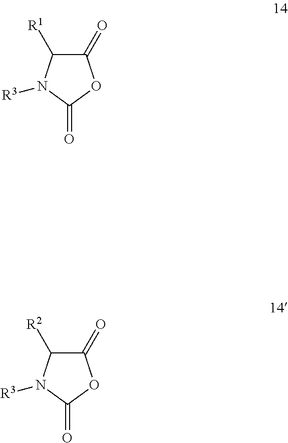

wherein R¹, R² and R³ are as defined above.

According to another particular embodiment, the present invention relates to a use as defined above, wherein at least one of the groups R¹ or R², comprises at least one protective group of carboxylic acid functions, amine functions, thiol functions, guanidine functions, amide functions and/or alcohol functions, in particular selected from tert-butyloxycarbonyl (Boc), benzyloxycarbonyl (Cbz) fluorenylmethyloxycarbonyl (Fmoc), alloc, tert-butyloxy (OtBu), formyl (For), 2,2,4,6,7-pentamethylhydrobenzofuran-5-sulfonyl (Pbf), 2,2,5,7,8-pentamethylchroman-6-sulfonyl (Pme), 4-methoxy-2,3,6-trimethylbenzenesulfonyl (Mtr), trityl (Trt), trifluoroacetyl, acetamidomethyl (Acm), and xanthyl (Xan).

The protective groups used according to the present invention are any protective group available to those skilled in the art (Greene's Protective Groups in Organic Synthesis, 4th edition, Wiley).

According to another particular embodiment, the present invention relates to a use as defined above, wherein the NCA compound is selected from:
alanine-NCA, arginine-NCA, asparagine-NCA, aspartic acid-NCA, cysteine-NCA, glutamine-NCA, glutamic acid-NCA, glycine-NCA, histidine-NCA, isoleucine-NCA, leucine-NCA, lysine-NCA, methionine-NCA, phenylalanine-NCA, proline-NCA, serine-NCA, threonine-NCA, tryptophane-NCA, tyrosine-NCA, valine-NCA, 1,2,3,4-tetrahydro-isoquinoline-3-carboxylic acid-NCA, (Tic-NCA), 2-amino-2-methylpropanoic acid-NCA (Aib-NCA), and norleucine-NCA (Nle-NCA),
said NCA compounds being optionally protected, on the carboxylic acid functions, amine functions, thiol functions, guanidine functions, amide functions and/or alcohol functions, by a protective group, in particular selected from tert-butyloxycarbonyl (Boc), benzyloxycarbonyl (Cbz) fluorenylmethyloxycarbonyl (Fmoc), alloc, tert-butyloxy (OtBu), formyl (For), 2,2,4,6,7-pentamethylhydrobenzofuran-5-sulfonyl (Pbf), 2,2,5,7, 8-pentamethylchroman-6-sulfonyl (Pmc), 4-methoxy-2,3,6-trimethylbenzenesulfonyl (Mtr), trityl (Trt), trifluoroacetyl, acetamidomethyl (Acm), and xanthyl (Xan).

The present invention also relates to a use as defined above, wherein the NCA compound is a UNCA compound, in particular a UNCA compound of the following structure:

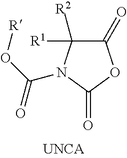

wherein R′ is selected from linear or branched C₁ to C₂₀ alkyl group, in particular a tert-butyl, or an aryl-methyl group, in particular a benzyl group or a fluorenylmethyl group,
wherein R¹ and R² are as defined above.

According to another particular embodiment, the present invention relates to a use as defined above, wherein the NCA compound is selected from the following structures:

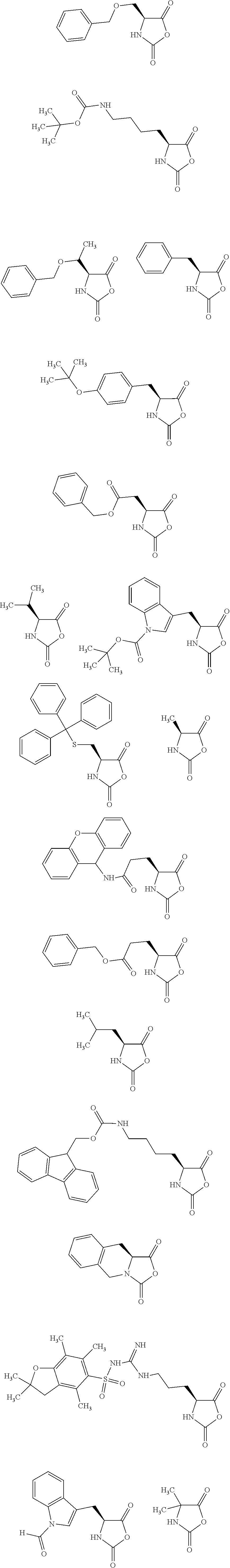

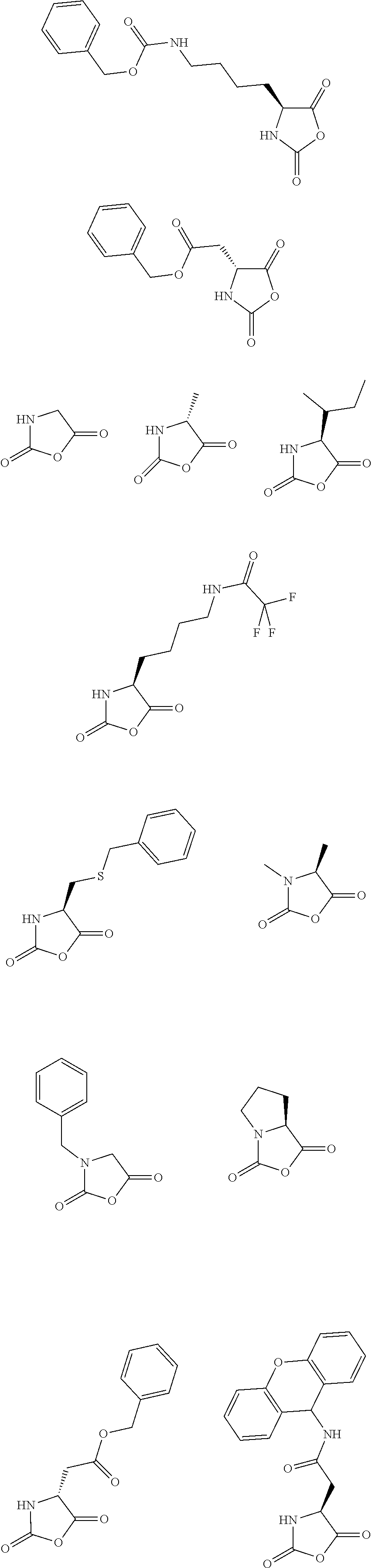

A second object of the present invention is a method for preparing an NCA compound, comprising:
a step of contacting a N-protected α-amino-acid compound,
with propane-phosphonic acid anhydride, and
optionally an organic base,
in an organic solvent,
   to obtain said compound NCA,
   wherein said α-amino-acid compound is N-protected on the α amine function by a linear or branched C₁ to C₂₀ C(O)—O-alkyl substituent, in particular by a -tert-butyloxycarbonyl group.

According to a particular embodiment, the present invention relates to a method as defined above, wherein the contact step comprises:
a stirring step at a temperature of 40 to 80° C., in the absence of said organic base.

According to another particular embodiment, the present invention relates to a method as defined above, wherein the contact step comprises:
a stirring step, especially at room temperature, in the presence of said organic base.

Alternatively, the method of the present invention can be implemented in a flow chemistry device.

According to another particular embodiment, the present invention therefore relates to a method as defined above, said method being implemented in continuous flow.

According to another particular embodiment, the present invention relates to a method as defined above, further comprising, after obtaining the NCA, a purification step by at least one aqueous wash.

Aqueous washing makes it possible to remove the propylphosphonic acid formed during the reaction.

According to another particular embodiment, the present invention relates to a method as defined above, further comprising a step of purifying the NCA compound, in particular by recrystallization.

According to another particular embodiment, the present invention relates to a method as defined above, wherein the contact step is carried out for a period of 1 to 24 hours, in particular about 2 hours.

The method according to the present invention generally allows to obtain a total conversion after 2 hours of reaction. However, the reaction time may be longer if the analysis of an aliquot demonstrates the presence of N-protected-α-amino-acid.

According to another particular embodiment, the present invention relates to a method as defined above, in the presence of an organic base, said organic base being a nitrogenous base, in particular selected from triethylamine, 1,8-diazabicyclo [5.4.0] undec-7-ene, diisopropylethylamine, N-dimethylaminopyridine, diisopropylethylamine or pyridine, preferably pyridine.

According to another particular embodiment, the present invention relates to a method as defined above, wherein the organic solvent is selected from ethyl acetate or dimethylformamide, in particular ethyl acetate.

According to another particular embodiment, the present invention relates to a method as defined above, wherein the propane-phosphonic acid anhydride is used in an amount of 1 to 4 molar equivalents relative to the N-protected α-amino-acid compound, in particular 2 molar equivalents.

According to another particular embodiment, the present invention relates to a method as defined above, in the presence of an organic base in an amount of 0.25 to 3 molar equivalents relative to the N-protected α-amino-acid compound, in particular 1 to 3 molar equivalents, preferably 3 molar equivalents.

According to another particular embodiment, the present invention relates to a method as defined above, in the presence of an organic base, wherein the base is pyridine at a rate of 3 molar equivalents relative to the N-protected α-amino-acid compound, wherein the propane-phosphonic acid anhydride is used in an amount of 2 molar equivalents relative to the N-protected α-amino-acid compound, and wherein the stirring step is carried out at room temperature.

According to another particular embodiment, the present invention relates to a method as defined above, wherein the propane-phosphonic acid anhydride is used in an amount of 4 molar equivalents relative to the N-protected α-amino-acid compound, and wherein the stirring step is carried out at a temperature of 50 to 80° C., in the absence of an organic base.

According to another particular embodiment, the present invention relates to a method for preparing a compound NCA, as defined above, comprising:
a step of contacting a N-protected α-amino-acid compound with:
propane-phosphonic acid anhydride, and
optionally an organic base, in particular a nitrogenous base, in particular selected from triethylamine, 1,8-diazabicyclo [5.4.0] undec-7-ene, diisopropylethylamine, N-dimethylaminopyridine, diisopropylethylamine or pyridine, preferably pyridine,
in an organic solvent, in particular selected from ethyl acetate or dimethylformamide,
to obtain said compound NCA,
and optionally:
   after obtaining the NCA, one purification step by at least one aqueous wash, and/or
   a step of purification of the NCA compound, in particular by recrystallization,
wherein said α-amino-acid compound is N-protected on the α amine function by a linear or branched C₁ to C₂₀ C(O)—O-alkyl substituent, in particular by a -tert-butyloxycarbonyl group.

According to another particular embodiment, the present invention relates to a method for preparing a NCA compound, as defined above, wherein the contact step comprises:
a stirring step at a temperature of 40 to 80° C., in the absence of said organic base, or
a stirring step, in particular at room temperature, in the presence of said organic base,
said method being optionally implemented in continuous flow.

According to another particular embodiment, the present invention relates to a method as defined above, wherein said NCA compound is of Formula 1-A, prepared from a N-protected α-amino-acid compound of Formula 2-A, according to the following reaction scheme:

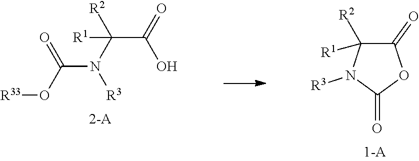

Form 1-A in which:
R¹ and R² independently represent:
   a hydrogen atom,
   a group chosen from:
      a linear or branched C₁ to C₂₀ alkyl,
      a linear or branched C₂ to C₂₀ alkenyl,
      C₃ to C₁₀ cycloalkyl,
      C₃ to C₁₀ heterocycloalkyl, wherein the heteroatom is selected from N, O, and S,
      aryle,
      C₁ to C₂₀ alkyl-aryl, in particular benzyl,
      heteroaryl, wherein the heteroatom is selected from N, O, and S, in particular 3-methylindole,
      C₁ to C₂₀ alkyl-heteroaryl, wherein the heteroatom is selected from N, O, and S,
      a halogen, in particular a fluorine atom,
   said alkyl, alkyl-aryl or alkyl-heteroaryl may be substituted on at least one carbon of the alkyl radical by one or more groups selected from:
      O—R⁴, wherein R⁴ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
      O—C(O)—R⁵, wherein R⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      C(O)—R⁶, wherein R⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      C(O)—O—R⁷, wherein R⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      NR⁸R⁹, wherein R⁸ and R⁹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      (NH)CNHR¹⁰, wherein R10 is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
      NR¹¹C(O)R¹², wherein R¹¹ and R¹² are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R¹² being in particular a protective group such as Boc, Cbz, Alloc or Fmoc,
      C(O)—NR¹³R¹⁴, wherein R¹³ and R¹⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      S—R¹⁵, wherein R¹⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
      a halogen, in particular selected from F, Cl, Br and I,
   said aryl, alkyl-aryl, heteroaryl and alkyl-heteroaryl may be substituted on the aromatic or heteroaromatic ring by one or more groups selected from:
      alkyl, C₁ to C₂₀, linear or branched,
      cycloalkyl, C₃ to C₁₀,
      heterocycloalkyl, C₃ to C₁₀,
         O—R¹⁶, wherein R¹⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
         O—C(O)—R¹⁷, wherein R¹⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
         C(O)—R¹⁸, wherein R¹⁸ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
         C(O)—O—R¹⁹, wherein R¹⁹ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
         NR²⁰R²¹, wherein R²⁰ and R²¹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
         (NH) CNHR²², wherein R²² is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
         NR²³C(O)R²⁴, wherein R²³ and R²⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R²⁴ being in particular a protective group such as Boc, Cbz, Alloc or Fmoc,
         C(O)—NR²⁵R²⁶, wherein R²⁵ and R²⁶ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀alkyl-heteroaryl,
         S—R²⁷, wherein R²⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
         a halogen, in particular selected from F, Cl, Br and I, R¹ and R² may form a cycle,
R³ represents:
   a hydrogen atom,
   a linear or branched C₁ to C₂₀ alkyl group,
      said group R³ may form a cycle with R¹ or R²,
      said compound of Formula 1 may be in the form of a solvate or a hydrate,
      said groups NR⁸R⁹, (NH)CNHR¹⁰, NR²⁰R²¹ and (NH)CNHR²², heteroaryl, alkyl-heteroaryl and/or heterocycloalkyl may be in a salified form,
      and Formula 2-A wherein R¹, R² and R³ are as defined above for Formula 1-A, and wherein R³³ represents a linear or branched C₁ to C₂₀ alkyl group, in particular a tert-butyl.

When the compound of Formula 1-A, or the compound of Formula 2-A, comprises a carbon atom, said carbon atom may be ¹³C,

when the compound of Formula 1-A, or the compound of Formula 2-A, comprises a fluorine atom, said fluorine atom may be ¹⁸F,
when the compound of Formula 1-A, or the compound of Formula 2-A, comprises a hydrogen atom, said hydrogen atom may be deuterium,
the asymmetric centers of said compound of Formula 1-A, and said compound of Formula 2-A, are of R or S configuration, or a mixture thereof.

The carboxylic acid function present in the compound of Formula 2-A is optionally in the form of carboxylate, especially in the form of a sodium salt, a potassium salt, or a lithium salt.

According to another particular embodiment, the present invention relates to a method as defined above, wherein the compound of Formula 1-A and the compound of Formula 2-A are such that:
R¹ and R² independently represent:
   a hydrogen atom,
   a group chosen from:
      a linear or branched C₁ to C₂₀ alkyl,
      C₃ to C₁₀ cycloalkyl,
      aryle,
      C₁ to C₂₀ alkyl-aryl, in particular benzyl,
      heteroaryl, wherein the heteroatom is selected from N, O, and S, in particular 3-methylindole,
      C₁ to C₂₀ alkyl-heteroaryl, wherein the heteroatom is selected from N, O, and S,
      a fluorine atom,
   said alkyl, alkyl-aryl or alkyl-heteroaryl may be substituted on at least one carbon of the alkyl radical by one or more groups selected from:
      O—R⁴, wherein R⁴ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
      NR⁸R⁹, wherein R⁸ and R⁹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
      (NH)CNHR¹⁰, wherein R¹⁰ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO 2, Pbf, Pmc, Mtr or Boc,
      NR¹¹C(O)R¹², wherein R¹¹ and R¹² are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R¹² being in particular a protective group such as Boc, Cbz, Alloc or Fmoc,
      S—R¹⁵, wherein R¹⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
   said aryl, alkyl-aryl, heteroaryl and alkyl-heteroaryl may be substituted on the aromatic or heteroaromatic ring by one or more groups selected from:
      alkyl, C₁ to C₂₀, linear or branched,
         O—R¹⁶, wherein R¹⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
         NR²⁰R²¹, wherein R²⁰ and R²¹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀alkyl-heteroaryl,
         (NH)CNHR²², wherein R²² is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
         NR²³C(O)R²⁴, wherein R²³ and R²⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R²⁴ being in particular a protective group such as Boc, Cbz, Alloc or Fmoc,
         S—R²⁷, wherein R²⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
         a halogen, in particular selected from F, Cl, Br and I,
      R¹ and R² may form a cycle,
R³ represents:
a hydrogen atom,
a linear or branched C₁ to C₂₀ alkyl group,
said group R³ may form a cycle with R¹ or R²,
R³³ represents a linear or branched C₁ to C₂₀ alkyl, in particular a tert-butyl,
said compound of Formula 1-A may be in the form of a solvate or hydrate,
said groups NR⁸R⁹, (NH)CNHR¹⁰, NR²⁰R²¹ and (NH)CNHR²², heteroaryl, alkyl-heteroaryl and/or heterocycloalkyl compound of Formula 1-A may be in a salified form,
   when the compound of Formula 1-A, or the compound of Formula 2-A, comprises a carbon atom, said carbon atom may be ¹³C,
   when the compound of Formula 1-A, or the compound of Formula 2-A, comprises a fluorine atom, said fluorine atom may be ¹⁸F,
   when the compound of Formula 1-A, or the compound of Formula 2-A, comprises a hydrogen atom, said hydrogen atom may be deuterium,
   the asymmetric centers of said compound of Formula 1-A, and said compound of Formula 2-A, are of R or S configuration, or a mixture thereof.

According to another particular embodiment, the present invention relates to a method as defined above, wherein the compound of Formula 1-A and the compound of Formula 2-A are such that:
R¹ and R² independently represent:
   a hydrogen atom,
   a group chosen from:
      a linear or branched C₁ to C₂₀ alkyl,
      C₁ to C₂₀ alkyl-aryl, in particular benzyl,
      heteroaryl, wherein the heteroatom is selected from N, O, and S, in particular 3-methylindole,
   said alkyl or alkyl-aryl may be substituted on at least one carbon of the alkyl radical by one or more groups selected from:
      O—R⁴, wherein R⁴ is selected from H, linear or branched C₁ to C₂₀ alkyl, and a protective group, in particular selected from tBDMS, t-butyl, benzyl, trityl and xanthyl,
      NR⁸R⁹, wherein R⁸ and R⁹ are independently selected from H, linear or branched C₁ to C₂₀ alkyl,
      (NH) CNHR¹⁰, wherein R¹⁰ is selected from H, linear or branched C₁ to C₂₀ alkyl, and a protective group, in particular NO₂, Pbf, Pmc, Mtr or Boc,
      NR¹¹C(O)R¹², wherein R¹¹ and R¹² are independently selected from H, linear or branched C₁ to C₂₀ alkyl, linear or branched C₁ to C₂₀ O-alkyl, linear or branched C₁ to C₂₀ O-alkyl-aryl, the radical —C(O)R¹² being in particular a protective group such as Boc, Cbz, Alloc or Fmoc,
      S—R¹⁵, wherein R¹⁵ is selected from H, linear or branched C₁ to C₂₀ alkyl and a protective group, in particular trityl or acetamidomethyl (Acm),
   said alkyl-aryl or heteroaryl may be substituted on the aromatic or heteroaromatic ring by one or more groups selected from:
      a linear or branched C₁ to C₂₀ alkyl,
      O—R¹⁶, wherein R¹⁶ is selected from H, linear or branched C₁ to C₂₀ alkyl and a protective group, in particular selected from tBDMS, t-butyl, benzyl, trityl and xanthyl,
      NR²⁰R²¹, wherein R²⁰ and R²¹ are independently selected from H, linear or branched C₁ to C₂₀ alkyl,
      (NH)CNHR²², wherein R²² is selected from H, linear or branched C₁ to C₂₀ alkyl, and a protective group, in particular NO₂, Pbf, Pmc, Mtr or Boc,
      NR²³C(O)R²⁴, wherein R²³ and R²⁴ are independently selected from H, linear or branched C₁ to C₂₀ alkyl, linear or branched C₁ to C₂₀ O-alkyl, linear or branched C₁ to C₂₀ O-alkyl-aryl, the radical —C(O)R²⁴ being in particular a protective group such as Boc, Cbz, Alloc or the Fmoc,
      S—R²⁷, wherein R²⁷ is selected from H, linear or branched C₁ to C₂₀ alkyl, and a protective group, in particular trityl or acetamidomethyl (Acm),
      a fluorine atom,
R³ represents:
   a hydrogen atom,
   a linear or branched C₁ to C₂₀ alkyl group,
said group R³ may form a cycle with R¹ or R²,
R³³ represents a linear or branched C₁ to C₂₀ alkyl group, in particular a tert-butyl,
said compound of Formula 1-A may be in the form of a solvate or hydrate,
said groups NR⁸R⁹, (NH) CNHR¹⁰, NR²⁰R²¹ and (NH) CNHR²², and/or heteroaryl compound of Formula 1-A may be in a salified form,
   the asymmetric centers of said compound of Formula 1-A, and said compound of Formula 2-A, are of R or S configuration, or a mixture thereof.

According to another particular embodiment, the present invention relates to a method as defined above, wherein the compound of Formula 1-A and the compound of Formula 2-A are such that:
R¹ and R² independently represent:
   a hydrogen atom,
   a group chosen from:
      a linear or branched C₁ to C₁₀ alkyl,
      3-methylindole,
   said alkyl may be substituted by one or more groups selected from:
      O—R⁴, wherein R⁴ is a t-butyl group,
      (NH)CNHR¹⁰, wherein R¹⁰ is selected from a protective group, in particular NO₂, Pbf, Pmc, Mtr or Boc,
      NR¹¹C(O)R¹², wherein R¹¹ is H, and the radical —C(O) R¹² is a protective group such as Boc, Cbz, Alloc or Fmoc,
      S—R¹⁵, wherein R¹⁵ is trityl or acetamidomethyl (Acm),
R³ represents:
   a hydrogen atom,
   a linear or branched C₁ to C₂₀ alkyl group,
      said group R³ may form a cycle with R¹ or R²,
R³³ represents a tert-butyl group,
   said compound of Formula 1-A may be in the form of a solvate or hydrate,
   said groups NR⁸R⁹, (NH)CNHR¹⁰, NR²⁰R²¹ and (NH) CNHR²² and/or 3-methylindole, of the compound of Formula 1-A may be in a salified form,
   the asymmetric centers of said compound of Formula 1-A, and said compound of Formula 2-A, are of R or S configuration, or a mixture thereof.

According to another particular embodiment, the present invention relates to a method as defined above, wherein R³ is a hydrogen atom.

According to another particular embodiment, the present invention relates to a method as defined above, wherein R¹ and R² are identical, and represent in particular a methyl.

According to another particular embodiment, the present invention relates to a method as defined above, wherein one of R¹ or R² is a hydrogen atom.

According to another particular embodiment, the present invention relates to a method as defined above, wherein R¹ or R² forms a cycle.

According to another particular embodiment, the present invention relates to a method as defined above, wherein at least one of the groups R¹ or R² comprises at least one protective group, carboxylic acid functions, amine functions, thiol functions, guanidine functions, amide functions and/or alcohol functions, in particular selected from tert-butyloxycarbonyl (Boc), benzyloxycarbonyl (Cbz) fluorenylmethyloxycarbonyl (Fmoc), alloc, tert-butyloxy (OtBu), formyl (For), 2,2,4,6,7-pentamethylhydrobenzofuran-5-sulfonyl (Pbf), 2,2,5,7,8-pentamethylchroman-6-sulfonyl (Pmc), 4-methoxy-2,3,6-trimethylbenzenesulfonyl (Mtr), trityl (Trt), trifluoroacetyl, acetamidomethyl (Acm), and xanthyl (Xan).

According to another particular embodiment, the present invention relates to a method as defined above, comprising:
a step of contacting a N-protected α-amino-acid compound,
   with propane-phosphonic acid anhydride, and
   optionally an organic base,
   in an organic solvent,
to obtain said compound NCA,
and optionally
   one purification step by at least one aqueous wash,
and optionally
   a step of purification of the NCA compound, in particular by recrystallization,
      wherein said α-amino-acid compound is N-protected on the amine function in a by a linear or branched C₁ to C₂₀ C(O)—O— alkyl substituent, in particular by a -tert-butyloxycarbonyl group.

According to another particular embodiment, the present invention relates to a method as defined above, wherein the α-amino-acid compound is selected from:
alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, 1,2,3,4-tetrahydroisoquinoline-3-carboxylic acid (Tic), 2-amino-2-methylpropanoic acid (Aib), and norleucine (Nle),
said α-amino-acid compound being N-protected on the α amine function by a linear or branched C₁ to C₂₀—C(O)—O-alkyl substituent, in particular by a -tert-butyloxycarbonyl group, said amino acids being optionally protected, on the side chain, on the carboxylic acid functions, amine functions, thiol functions, guanidine functions, amide functions and/or alcohol functions, by a protective group, in particular selected from tert-butyloxycarbonyl (Boc), benzyloxycarbonyl (Cbz) fluorenylmethyloxycarbonyl (Fmoc), alloc, tert-butyloxy (OtBu), formyl (For), 2,2,4,6,7-pentamethylhydrobenzofuran-5-sulfonyl (Pbf), 2,2,5,7,8-pentamethylchroman-6-sulfonyl (Pme), 4-methoxy-2,3,6-trimethylbenzenesulfonyl (Mtr), trityl (Trt), trifluoroacetyl, acetamidomethyl (Acm), and xanthyl (Xan).

The present invention also relates to a method as defined above, wherein the NCA compound is a UNCA compound, in particular a UNCA compound of the following structure:

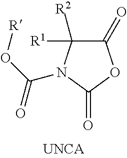

wherein R′ is selected from a linear or branched C₁ to C₂₀ alkyl group, in particular a tert-butyl, or an aryl-methyl group, in particular a benzyl group or a fluorenylmethyl group, wherein R¹ and R² are as defined above.

A third object of the present invention is an NCA compound, as obtained by the method as defined above.

The method for preparing a NCA compound according to the invention provides NCA compounds of excellent purities, and devoid of phosgene decomposition products, diphosgene decomposition products and triphosgene decomposition products, in particular devoid of hydrochloric acid. The method also avoids contamination observed when the NCA compound is prepared under nitrosation conditions.

These breakdown products can harm certain uses of NCA compounds, especially in the synthesis of a peptide, where the presence of hydrochloric acid residues for example is troublesome.

However, such compounds according to the present invention can be salified, after their isolation, during a salification step wherein the NCA compound is put in the presence of an acid for example such as hydrochloric acid or triflic acid, non-nucleophilic. However, this is the salification of a salivable function on the side chain, and not of the nitrogen of the NCA cycle whose salified forms are unstable.

A fourth object of the present invention is a novel NCA compound, selected from the following structures:

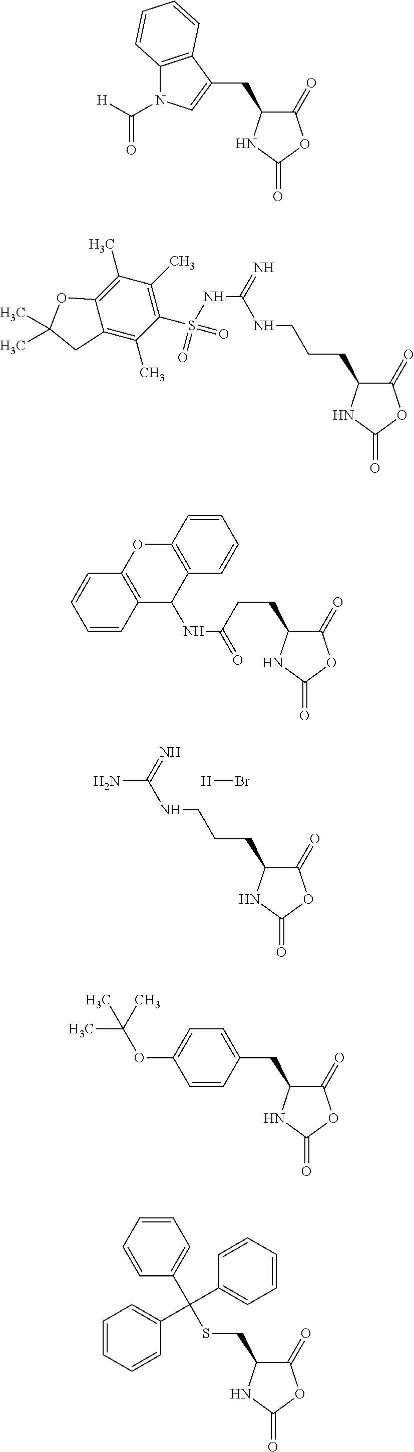

The invention also relates to a novel NCA compound of the following structure:

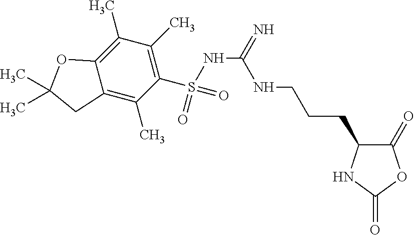 [SMILES: Cc1c(C)c(S(=O)(=O)NC(=N)NCCC[C@@H]2NC(=O)OC2=O)c(C)c2c1OC(C)(C)C2]

According to another particular embodiment, the present invention relates to a novel NCA compound as defined below, selected from:

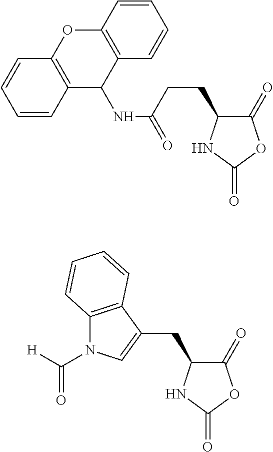 [SMILES: O=C(CC[C@@H]1NC(=O)OC1=O)NC1c2ccccc2Oc2ccccc21 ; O=Cn1cc(C[C@@H]2NC(=O)OC2=O)c2ccccc21]

A fifth object of the present invention is a solution comprising a NCA compound prepared according to the method as defined above, or a NCA compound as defined above, said solution being devoid of phosgene decomposition products, diphosgene decomposition products and triphosgene decomposition products, in particular devoid of hydrochloric acid.

A sixth object of the present invention is the use of an NCA compound prepared according to the method as defined above, or a NCA compound as defined below, in the preparation of a polypeptide, oligopeptide or dendrimer.

A seventh object of the present invention is a method for preparing a polypeptide, comprising a step of contacting a NCA compound prepared according to the method as defined above, or an NCA compound as defined above, with a polymerization initiator, in particular selected from amines, in particular arginine.

An eighth object of the present invention is a polypeptide, as obtained by the method for preparing a polypeptide as defined above.

A ninth object of the present invention is a method for preparing a UNCA compound, comprising a contact step of an NCA compound prepared according to the method as defined above, or an NCA compound as defined above, with:
either a reagent for introducing a tert-butyloxycarbonyl protective group, in particular selected from (Boc)₂O and Boc-OSu,
to obtain a UNCA compound protected on the nitrogen atom of the NCA ring by a -Boc group,
either a reagent for introducing a benzyloxycarbonyl protective group, in particular selected from Cbz-Cl and Cbz-OSu,
to obtain a UNCA compound protected on the nitrogen atom of the NCA ring by a -Cbz group,
or a reagent for introducing a fluorenylmethyloxycarbonyl protective group, in particular selected from Fmoc-Cl and Fmoc-OSu,
to obtain a UNCA compound protected on the nitrogen atom of the NCA ring by a -Fmoc group.

According to this embodiment, the starting NCA compound comprises an NCA ring in which the nitrogen atom is in the form —NH, allowing to introduce a substituent.

The following examples and figures illustrate the invention, without limiting its scope.

FIG. 1 show the analytical data obtained for the compound (L) Trp (Boc)-NCA of Example 2.

FIG. 1A shows the NMR spectrum of the proton,

FIG. 1B shows the LC chromatogram, and

FIG. 1C shows the mass spectrum.

FIG. 2 show an analysis by chiral HPLC on diastereoisomers obtained according to Example 22.

FIG. 2A shows the LC chromatogram of the racemic mixture,

FIG. 2B shows the LC chromatogram of the diastereomer S, S, and

FIG. 2C shows the LC chromatogram of the diastereoisomer R,S.

### EXAMPLES

Materials and Methods

Reagents and solvents were used as procured from commercial suppliers, and without further purification. The ¹³C and ¹H NMR spectra in DMSO-d₆ and CDCl₃ were performed on a Bruker AVANCE 600 MHz spectrometer, equipped with a BBFO helium cryoprobe at 298 K. Chemical shifts (δ) are reported in parts per million using non-deuterated residual solvents as internal references. The spectra were processed and visualized with Topspin 3.2 (Bruker Biospin).

LC/MS analyses were performed using a 25×4.6 mm C18 Chromolith Flash inverted phase column. A flow rate of 3 ml/min and a gradient of (0 to 100%) of B over 2.5 min were used. Voting agent A: water/0.1% HCO₂H; eluent B: acetonitrile/0.1% HCO₂H. UV detection was performed at 214 nm. The electrospray mass spectra were acquired at a solvent rate of 200 μL/min. Nitrogen was used for both nebulizing and drying gas. Data were obtained in a scanning mode ranging from 100 to 1000 m/z or from 250 to 1500 m/z at intervals of 0.7 s.

### Example 1: General Procedure for the Synthesis of NCA Compounds—in the Presence of Pyridine

Boc-AA-OH (1 eq) was solubilized in ethyl acetate or dimethylformamide. Pyridine (3 eq) was added, followed by T3P®/AcOEt 50% (2 eq), drip. The reaction mixture was stirred at room temperature for 2 hours, and the progress of the reaction was analyzed by HPLC and LC/MS.

The reaction mixture was diluted in ethyl acetate and washed twice with cold water and then with an aqueous solution saturated with NaCl. The organic phase was dried on anhydrous magnesium sulphate, filtered and concentrated under vacuum. The expected product was purified by recrystallization.

The compounds of Examples 2 to 18 and 24 to 34 below were prepared according to this general procedure, using ethyl acetate as reaction solvent and on a scale of 1 gram of starting amino acid.

### Example 2: Preparation of (L)Trp(Boc)-NCA

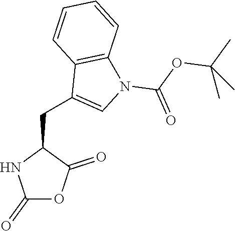 [SMILES: CC(C)(C)OC(=O)n1cc(C[C@@H]2NC(=O)OC2=O)c2ccccc21]

Yield 81%

White solid, mp 152-153° C., [α]20_{D} (c 1.00, CH₂Cl₂)=−49.1°, ¹H NMR (600 MHz, CDCl₃) δ=8.04 (brd, J=8 Hz, 1H), 7.43-7.37 (m, 2H), 7.29-7.22 (m, 1H), 7.20-7.13 (m, 1H), 6.36 (brs, 1H), 4.50-4.44 (dd, J=3.64, 8.64 Hz, 1H), 3.33-3.25 (dd, J=3.64, 14.69 Hz, 1H), 3.03-3.93 (dd, J=8.64, 14.69 Hz, 1H), 1.57 (s, 9H), ¹³C NMR (125 MHz, CDCl₃) δ=168.8, 152.0, 149.4, 135.5, 129.3, 125.0, 124.8, 122.9, 118.4, 115.6, 113.1, 84.3, 57.6, 28.1, 27.7, LC/MS (ESI⁺) t_{R}1.81 min, m/z 331 [M+H]+ m/z 231 [M+H−Boc]⁺.

### Example 3: Preparation of (L)Val-NCA

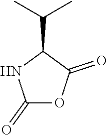

Recrystallized in an AcOEt/hexane mixture

Yield 78%

White solid, mp 62-63° C., ¹H NMR (600 MHz, CDCl₃) δ=6.95 (brs, 1H), 4.28-4.21 (d, J=4.20 Hz, 1H), 2.34-2.20 (m, 1H), 1.14-1.08 (d, J=6.97 Hz, 3H), 1.07 (d, J=6.97 Hz, 3H), ¹³C NMR (125 MHz, CDCl₃) δ=168.7, 153.4, 63.1, 30.7, 18.2, 16.6, LC/MS (ESI+) t R 1.68 min, m/z 144 [M+H]⁺.

### Example 4: Preparation of (L)Tyr(OtBu)-NCA

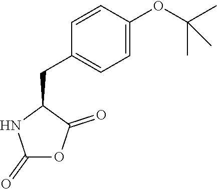 [SMILES: CC(C)(C)Oc1ccc(C[C@@H]2NC(=O)OC2=O)cc1]

Yield 90%

White solid, mp 108-109° C., [α]19_{D} (c 1.00, CH₂Cl₂)=−78.1°, ¹H NMR (600 MHz, CDCl₃) δ=7.00-6.96 (d, J=8.44 Hz, 2H), 6.89-6.86 (d, J=8.44 Hz, 2H), 5.67-5.55 (m, 1H), 4.42-4.37 (ddd, J=0.83, 4.06, 8.86 Hz, 1H), 3.18-3.13 (dd, J=4.06, 14.15 Hz, 1H), 2.86-2.80 (dd, J=8.86, 14.15, 1H), 1.24 (s, 9H), ¹³C NMR (125 MHz, CDCl₃) δ=168.5, 155.3, 151.3, 129.6, 128.5, 124.7, 78.8, 58.8, 37.3, 28.8, LC t_{R} 1.53 min.

### Example 5: Preparation of (L)Phe-NCA

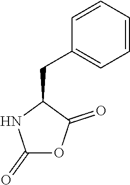 [SMILES: O=C1N[C@@H](Cc2ccccc2)C(=O)O1]

Recrystallized in an AcOEt/hexane mixture

Yield 87%

White solid, mp 88-89° C., [α]18 D (c 1.00, CH₂Cl₂)=−94.6, ¹H NMR (600 MHz, CDCl₃) δ=7.35-7.25 (m, 3H), 7.18-7.12 (m, 2H), 6.45-6.09 (m, 1H), 4.53-4.47 (dd, J=4.21, 8.24 Hz, 1H), 3.27-3.19 (m, 1H), 3.02-2.94 (dd, J=8.24, 14.29, 1H). ¹³C NMR (125 MHz, CDCl₃) δ=168.6, 151.8, 133.8, 129.2, 128.0, 58.8, 37.8, LC t_{R} 2.25 min.

### Example 6: Preparation of (L)Asp(OBzl)-NCA

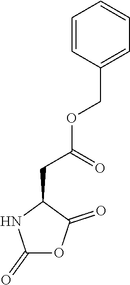 [SMILES: O=C(C[C@@H]1NC(=O)OC1=O)OCc1ccccc1]

Recrystallized in an AcOEt/hexane mixture

Yield 87%

White solid, mp 103-104° C., [α]19 D (c 1.00, CH2Cl2)=−42.3°, ¹H NMR (600 MHz, DMSO-d₆) δ=8.99 (s, 1H), 7.40-7.30 (m, 5H), 5.13 (s, 2H), 4.70-4.68 (t, J=4.57 Hz, 1H), 3.10-3.04 (dd, J=5.05, 17.86 Hz, 1H), 2.93-2.87 (dd, J=4.35, 17.86 Hz, 1H), ¹³C NMR (125 MHz, DMSO-d₆) δ=171.4, 169.7, 152.6, 136.0, 128.9, 128.6, 128.5, 66.7, 54.0, 35.1, LC t_{R} 2.55 min.

### Example 7: Preparation of (L)Thr(OBzl)-NCA

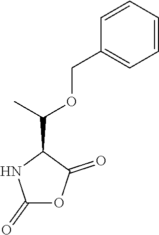 [SMILES: CC(OCc1ccccc1)[C@@H]1NC(=O)OC1=O]

Recrystallized in an AcOEt/hexane mixture

Yield 81%

White solid, mp 120-121° C., ¹H NMR (600 MHz, CDCl₃) δ=7.27-7.14 (m, 5H), 6.41 (brd, J=9.86 Hz, 1H), 4.53-4.50 (d, J=11.53 Hz, 1H), 4.35-4.32 (d, J=11.53 Hz, 1H), 4.09-4.06 (d, J=4.97 Hz, 1H), 3.84-3.78 (m, 1H), 1.25-1.23 (d, J=6.27 Hz, 3H), ¹³C NMR (125 MHz, CDCl₃) δ=167.5, 152.5, 152.5, 136.9, 128.5, 128.1, 127.9, 127.8, 73.1, 71.3, 62.7, 15.9, LC t_{R} 1.40 min.

### Example 8: Preparation of (L)Gln(Xan)-NCA

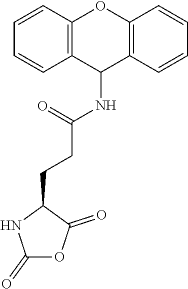 [SMILES: O=C(CC[C@@H]1NC(=O)OC1=O)NC1c2ccccc2Oc2ccccc21]

Yield 80%

White solid, mp 156-157° C., ¹H NMR (600 MHz, DMSO-d₆) δ=9.08 (s, 1H), 8.90-8.85 (d, J=8.80 Hz, 1H), 7.39-7.35 (m, 2H), 7.35-7.31 (m, 2H), 7.17-7.11 (m, 4H), 6.30-6.26 (d, J=8.81 Hz, 1H), 4.55-4.50 (t, J=5.92 Hz, 1H), 2.34-2.22 (m, 2H), 2.11-2.02 (m, 1H), 2.02-1.93 (m, 1H), ¹³C NMR (125 MHz, DMSO-d₆) δ=171.9, 170.8, 152.3, 150.9, 129.6, 129.5, 129.4, 123.9, 122.2, 122.2, 116.5, 56.9, 42.8, 30.5, 27.3, LC t_{R} 1.57 min.

### Example 9: Preparation of (L)Ala-NCA

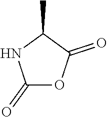

Recrystallized in an AcOEt/hexane mixture

Yield 76%

White solid, mp 84-85° C., ¹H NMR (600 MHz, CDCl₃) δ=6.41 (m, 1H), 4.33-4.26 (dd, J=7.07, 14.06 Hz, 1H), 1.47-1.45 (d, J=7.07 Hz, 3H), ¹³C NMR (125 MHz, CDCl₃) δ=170.0, 152.0, 53.3, 17.7.

### Example 10: Preparation of (L)Ser(OBzl)-NCA

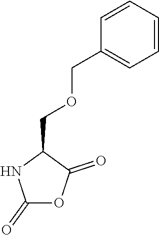 [SMILES: O=C1N[C@@H](COCc2ccccc2)C(=O)O1]

Recrystallized in an AcOEt/hexane mixture

Yield 77%

White solid, mp 65-66° C., ¹H NMR (600 MHz, DMSO-d₆) δ=9.10 (s, 1H), 7.40-7.17 (m, 6H), 4.66 (s, 1H), 4.59-4.48 (m, 2H), 3.81-3.71 (d, J=11.09 Hz, 1H), 3.67-3.60 (d, J=11.09 Hz, 1H), ¹³C NMR (125 MHz, DMSO-d₆) δ=170.4, 152.7, 138.0, 128.8, 128.1, 127.8, 72.9, 68.1, 58.8, LC t_{R} 1.36 min.

### Example 11: Preparation of (L) Glu(OBzl)-NCA

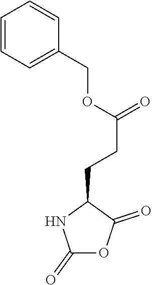 [SMILES: O=C(CC[C@@H]1NC(=O)OC1=O)OCc1ccccc1]

Recrystallized in an AcOEt/hexane mixture

Yield 78%

White solid, mp 110-111° C., [α]19_{D} (c 1.00, CH2Cl2)=−13.6°, ¹H NMR (600 MHz, DMSO-d₆) δ=9.08 (s, 1H), 7.40-7.29 (m, 5H), 5.10 (s, 2H), 4.49-4.44 (m, 1H), 2.54-2.48 (m, 2H), 2.10-2.00 (m, 1H), 1.99-1.89 (m, 1H), ¹³C NMR (125 MHz, DMSO-d₆) δ=172.1, 171.7, 152.3, 136.4, 128.9, 128.5, 128.4, 128.4, 66.1, 56.6, 29.5, 26.8, LC t_{R} 1.48 min.

### Example 12: Preparation of (L)Cys(Trt)-NCA

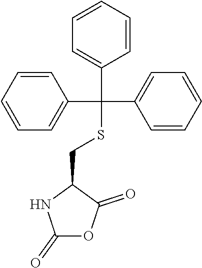 [SMILES: O=C1N[C@@H](CSC(c2ccccc2)(c2ccccc2)c2ccccc2)C(=O)O1]

Yield 82%

White solid, mp 122-123° C., ¹H NMR (600 MHz, DMSO-d₆) δ=9.16 (s, 1H), 7.39-7.23 (m, 18H), 4.42-4.39 (t, J=5.16 Hz, 1H), 2.53-2.50 (dd, J=2.37, 5.16 Hz, 2H), ¹³C NMR (125 MHz, DMSO-d₆) δ=170.3, 152.0, 144.2, 129.4, 128.6, 127.4, 66.7, 56.8, 32.9, LC t_{R} 2.00 min.

### Example 13: Preparation of (L)Lys (Boc)-NCA

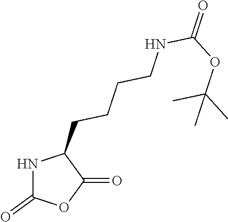 [SMILES: CC(C)(C)OC(=O)NCCCC[C@@H]1NC(=O)OC1=O]

Recrystallized in an AcOEt/hexane mixture

Yield 85%

White solid, mp 142-143° C., [α]19_{D} (DCM) [c=1]=−37.0°, ¹H NMR (600 MHz, DMSO-d₆) δ=9.05 (s, 1H), 6.81-6.72 (m, 1H), 4.46-4.38 (t, J=6.14 Hz, 1H), 2.94-2.85 (dd, J=6.14, 12.63 Hz, 2H), 1.76-1.68 (m, 1H), 1.67-1.59 (m, 1H), 1.42-1.30 (m, 12H), 1.30-1.21 (m, 1H), ¹³C NMR (125 MHz, DMSO-d₆) δ=172.1, 156.0, 152.4, 77.8, 57.4, 31.1, 29.3, 28.7, 22.0, LC t_{R} 1.39 min.

### Example 14: Preparation of (L)Tic-NCA

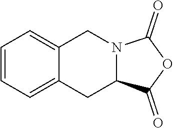 [SMILES: O=C1OC(=O)N2Cc3ccccc3C[C@H]12]

Recrystallized in an AcOEt/hexane mixture

Yield 76%

White solid, mp 166-167° C., ¹H NMR (600 MHz, DMSO-d₆) δ=7.34-7.18 (m, 4H), 4.81-4.74 (d, J=16.60 Hz, 1H), 4.64-4.60 (dd, J=5.13, 11.62 Hz, 1H), 4.49-4.43 (d, J=16.60 Hz, 1H), 3.25-3.18 (dd, J=11.62, 15.31 Hz, 1H), 3.18-3.12 (dd, J=5.13, 15.31 Hz, 1H), ¹³C NMR (125 MHz, DMSO-d₆) δ=170.1, 151.0, 131.6, 130.8, 129.8, 127.3, 127.3, 127.0, 54.6, 42.3, 29.2, LC t_{R} 1.39 min.

### Example 15: Preparation of (L)Leu-NCA

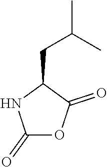

Recrystallized in an AcOEt/hexane mixture

Yield 86%

White solid, mp 66-67° C., ¹H NMR (600 MHz, DMSO-d₆) δ=9.11 (s, 1H), 4.49-4.41 (dd, J=5.45, 8.77 Hz, 1H), 1.80-1.67 (m, 1H), 1.63-1.51 (m, 2H), 0.94-0.83 (t, J=7.78 Hz, 6H), ¹³C NMR (125 MHz, DMSO-d₆) δ=172.4, 152.4, 56.0, 40.5, 24.5, 23.1, 21.7, LC t_{R} 1.18 min.

### Example 16: Preparation of (L)Lys (Fmoc)-NCA

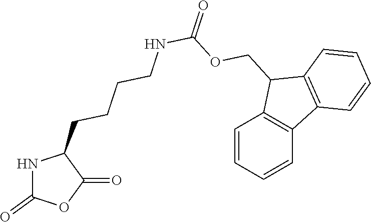 [SMILES: O=C(NCCCC[C@@H]1NC(=O)OC1=O)OCC1c2ccccc2-c2ccccc21]

Recrystallized in an AcOEt/hexane mixture

Yield 88%

White solid, mp 101-102° C., [α]20_{D}=−41.6 (c 1.00, CH₂Cl₂), ¹H NMR (600 MHz, DMSO-d₆) δ=9.07 (s, 1H), 7.88-7.87 (d, J=7.52 Hz, 2H), 7.68-7.67 (d, J=7α.53, 2H), 7.42-7.39 (dd, J=7.53, 14.85 Hz, 2H), 7.34-7.31 (dd, J=7.53, 14.85 Hz, 2H), 7.27 (m, 1H), 4.43 (m, 1H), 4.30 (m, 2H), 4.22 (m, 1H), 2.98 (m, 2H), 1.73 (m, 1H), 1.66 (m, 1H), 1.49-1.25 (m, 6H), ¹³C NMR (125 MHz, DMSO-d₆) δ=172.1, 156.5, 152.4, 144.4, 141.2, 128.0, 127.5, 125.5, 120.5, 65.6, 57.4, 47.2, 31.1, 29.2, 22.0, LC t_{R} 1.57 min.

### Example 17: Preparation of (L)Arg(Pbf)-NCA

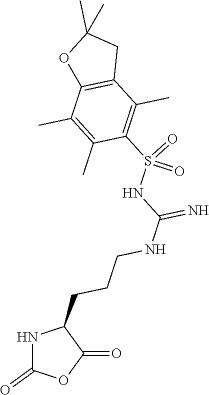 [SMILES: Cc1c(C)c(S(=O)(=O)NC(=N)NCCC[C@@H]2NC(=O)OC2=O)c(C)c2c1OC(C)(C)C2]

Recrystallized in an AcOEt/hexane mixture

Yield 86%

White solid, mp 122-123° C., [α]¹⁹_{D} (c 1.00, CH₂Cl₂)=−17.3°, ¹H NMR (600 MHz, CDCl₃) δ=7.71 (s, 1H), 6.28 (s, 3H), 4.28 (m, 1H), 3.64 (m, 1H), 2.84 (s, 2H), 2.42 (s, 3H), 2.35 (s, 3H), 1.97 (s, 3H), 1.84 (m, 1H), 1.75 (m, 2H), 1.65-1.45 (m, 3H), 1.35 (s, 9H), ¹³C NMR (125 MHz, CDCl₃) δ=170.5, 159.0, 156.4, 152.6, 138.2, 132.2, 124.9, 117.7, 86.6, 67.9, 57.2, 43.1, 28.5, LC t_{R} 1.56 min.

### Example 18: Preparation of (D)-Asp(OBzl)-NCA

Yield 79%

[α]¹⁹_{D} (c 1.00, CH₂Cl₂)+42.6°.

### Example 19: Setting Up an Operating Unit

(L)Leu-NCA from 10 g Boc-(L)Leu-OH

Boc-(L)leu-OH (10 g) was solubilized in ethyl acetate (300 ml). T3P® (2 equivalents), dissolved in ethyl acetate was added dropwise followed by pyridine (3 equivalents). The reaction mixture was stirred for 1 hour at room temperature. Water/ice (300 ml) was added, and the organic phase was recovered, washed 2 times with a cooled NaCl saturated aqueous solution (2×300 ml), dried over MgSO₄, filtered and concentrated under vacuum. Hexane (50 ml) was added to the oily residue, resulting in crystallization. The expected product was recrystallized in an AcOEt/hexane mixture, to obtain (L) Leu-NCA (5.9 g), which was stored under argon at −20° C.

Yield 86%.

(L)Phe-NCA from 20 g of Boc-(L)Phe-OH

Boc-(L)Phe-OH (20 g) was solubilized in ethyl acetate (1500 ml). T3P® (2 equivalents), dissolved in ethyl acetate was added dropwise followed by pyridine (3 equivalents). The reaction mixture was stirred for 1 hour at room temperature. Water/ice (1000 ml) was added, and the organic phase was recovered, washed 2 times with a cooled NaCl-saturated aqueous solution (2×500 ml), dried over MgSO₄, filtered and concentrated under vacuum. Hexane (100 ml) was added to the oily residue, resulting in crystallization. The expected product was recrystallized in an AcOEt/hexane mixture, to obtain (L) Phe-NCA (11.4 g), which was stored under argon at −20° C. Yield 79%.

(L)Glu(OBzl)-NCA from 25 g of Boc-(L)Glu(OBzl)-OH

Boc-(L)Glu(OBzl)-OH (25 g) was solubilized in ethyl acetate (1500 ml). T3P® (2 equivalents), dissolved in ethyl acetate was added dropwise followed by pyridine (3 equivalents). The reaction mixture was stirred for 1 hour at room temperature. Water/ice (1000 ml) was added, and the organic phase was recovered, washed 2 times with a cooled NaCl-saturated aqueous solution (2×500 ml), dried over MgSO₄, filtered and concentrated under vacuum. Hexane (100 ml) was added to the oily residue, resulting in crystallization. The expected product was recrystallized in an AcOEt/hexane mixture, to obtain (L) Glu (OBzl)-NCA (15 g), which was stored under argon at −20° C. Yield 79%.

(L)Ala-NCA from 50 g Boc-(L)Ala-OH

Boc-(L)Ala-OH (50 g) was solubilized in ethyl acetate (1500 ml). T3P® (2 equivalents), dissolved in ethyl acetate was added dropwise followed by pyridine (3 equivalents). The reaction mixture was stirred for 1 hour at room temperature. Water/ice (1000 ml) was added, and the organic phase was recovered, washed 2 times with a cooled NaCl-saturated aqueous solution (2×500 ml), dried over MgSO₄, filtered and concentrated under vacuum. Hexane (100 ml) was added to the oily residue, resulting in crystallization. The expected product was recrystallized in an AcOEt/hexane mixture, to obtain (L) Ala-NCA (22 g), which was stored under argon at −20° C. Yield 73%.

(L)Lys-NCA from 10 g Boc-(L)Lys-OH

Boc-(L)Lys-(Boc)-OH (10 g) was solubilized in ethyl acetate (300 ml). T3P® (2 equivalents), dissolved in ethyl acetate was added dropwise followed by pyridine (3 equivalents). The reaction mixture was stirred for 1 hour at room temperature. Water/ice (300 ml) was added, and the organic phase was recovered, washed 2 times with a cooled NaCl saturated aqueous solution (2×300 ml), dried over MgSO₄, filtered and concentrated under vacuum. Hexane (50 ml) was added to the oily residue, resulting in crystallization. The expected product was recrystallized in an AcOEt/hexane mixture, to obtain (L)Lys-(Boc)-NCA (6.3 g), which was stored under argon at −20° C. Yield 80%.

### Example 20: Preparation of (L)Tyr(OtBu)-NCA—Use of Different Bases

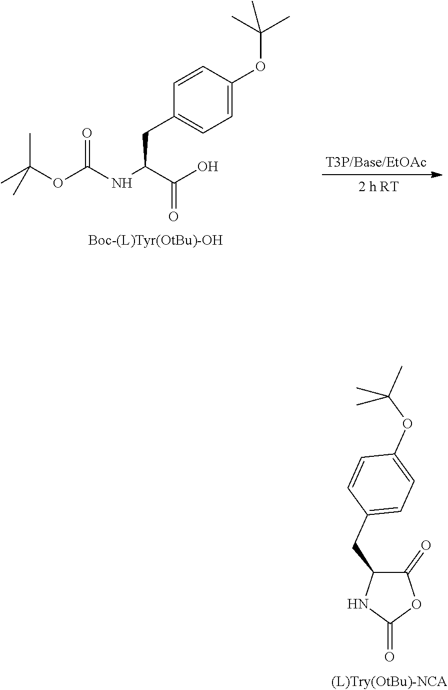 [SMILES: CC(C)(C)OC(=O)N[C@@H](Cc1ccc(OC(C)(C)C)cc1)C(=O)O ; CC(C)(C)Oc1ccc(C[C@@H]2NC(=O)OC2=O)cc1]

The general procedure of Example 1 was used, replacing pyridine with bases Et₃N, iPr₂EtN or DBU. After 2 hours of stirring at room temperature in ethyl acetate, the expected NCA product was formed in a majority manner, as analyzed by LC.

| Test | Base | Yield (%) |
|---|---|---|
| 1 | Et₃N | 69 |
| 2 | iPr₂EtN | 79 |
| 3 | DBU | 63 |

### Example 21: Preparation of NCA-(L)-Arg(Pbf)—in Absence of Base

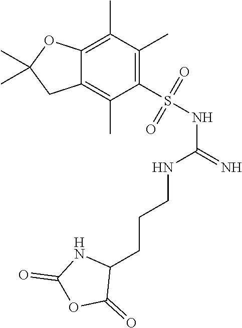 [SMILES: Cc1c(C)c(S(=O)(=O)NC(=N)NCCCC2NC(=O)OC2=O)c(C)c2c1OC(C)(C)C2]

Boc-(L)-Arg(Pbf)-OH (1 eq) was solubilized in ethyl acetate or dimethylformamide. T3P®/AcOEt 50% (4 eq) was added drop by drop. The reaction mixture was stirred at 70° C. for 2 hours, and the progress of the reaction was analyzed by HPLC and LC/MS. The reaction mixture was diluted in ethyl acetate and washed twice with cold water and then with an aqueous solution saturated with NaCl. The organic phase was dried on anhydrous magnesium sulphate, filtered and concentrated under vacuum. The expected product was purified by recrystallization. LC t_{R} 1.58 min.

### Example 22: Study of Stereochemistry

This test aims to confirm that the method according to the present invention is not racemic and isolates NCA compounds with retention of enantiomeric excess. The compounds (L) Asp (OBzl)-NCA and (D) Asp (OBzl)-NCA were prepared according to the procedure of Example 1, from Boc-(L) Asp (OBzl) —OH and Boc (D) Asp (OBzl) —OH respectively. Dimethylformamide was used as a reaction solvent.

The NCA compounds thus obtained were then reacted with (S)-1-(4-methoxyphenyl) ethane-1-amine to obtain diastereoisomers S, S and R, S.

An analysis by HPLC (Colone Zorbax SB-C18 50*2.1 mm 1.8 μm; flow rate 0.5 mL/min with a gradient (0-100%) B over 25 min; eluent A: water/0.1% HCO₂H; eluent B: acetonitrile/0.1% HCO₂H. UV detection was performed at 214 nm).

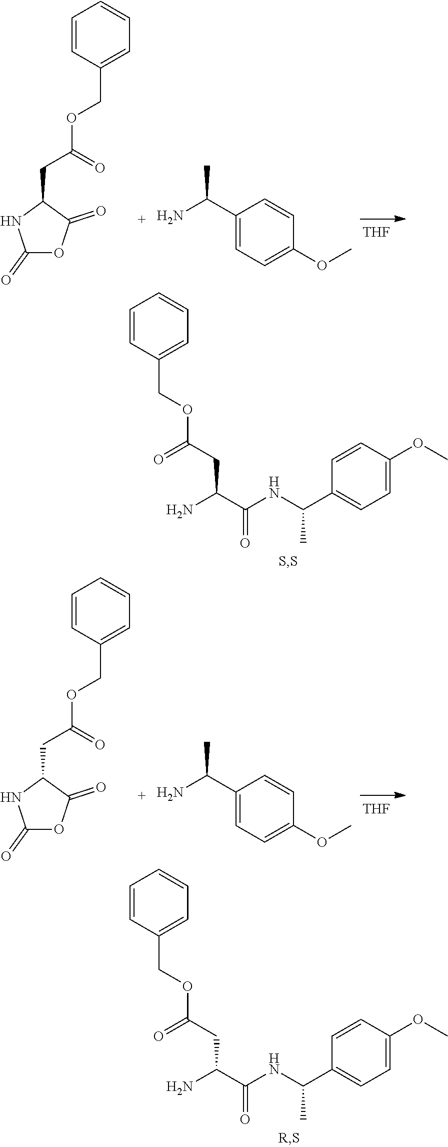

FIG. 2 shows the chromatograms thus obtained. Only one peak was observed in both cases (FIGS. 2B and 2C), confirming the retention of stereochemistry during the process. This result was also confirmed by ^{1H} NMR analysis of S,S and R,S diastereomers.

### Example 23: Comparative Example—DCC Versus T3P®

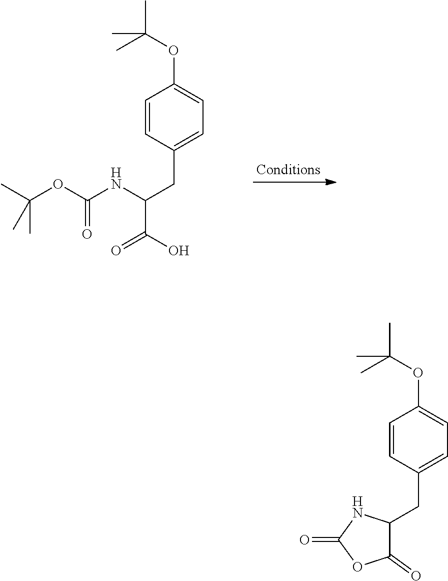 [SMILES: CC(C)(C)OC(=O)NC(Cc1ccc(OC(C)(C)C)cc1)C(=O)O ; CC(C)(C)Oc1ccc(CC2NC(=O)OC2=O)cc1]

| Test | Conditions | Outcome (assessed by HPLC) |
|---|---|---|
| 1 | DCC/Pyridine, 30 minutes | NCA traces * |
| | | predominant starting amino acid |
| 2 | DCC, 30 minutes | NCA traces |
| | | predominant starting amino acid |
| 3 | T₃P ®/Pyridine, | predominant NCA |
| | 30 minutes | Traces of starting amino acid |

* “traces of NCA” means <3% conversion.

In addition, the coupling agents HATU (2 equivalents) and BOP (2 equivalents) were also tested, in the presence of pyridine, with the same results as those obtained in the case of DCC, i.e. only traces of NCA were observed.

### Example 24: Preparation of Gly-NCA

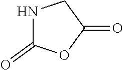

Recrystallized in an AcOEt/hexane mixture

Yield 80%

White solid, mp>200° C.

### Example 25: Preparation of (D)Ala-NCA

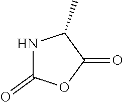

Recrystallized in an AcOEt/hexane mixture

Yield 80%

White solid, mp 112-113° C., [α]²⁰_{D}=−4 (c=1.00, CH₂Cl₂).

### Example 26: Preparation of (L)Ile-NCA

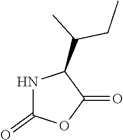

Recrystallized in an AcOEt/hexane mixture

Yield 86%

White solid, mp 66-68° C., [α]20_{D}=−29 (c=1.00, CH₂Cl₂).

### Example 27: Preparation of (L)Lys (Tfa)-NCA

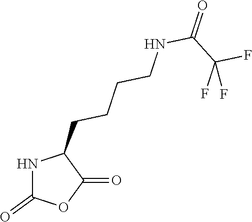 [SMILES: O=C1N[C@@H](CCCCNC(=O)C(F)(F)F)C(=O)O1]

Recrystallized in an AcOEt/hexane mixture

Yield 84%

White solid, mp 92-93° C., [α]²⁰_{D}=−31 (c=1.00, CH₂Cl₂).

### Example 28: Preparation of (L)Cys (S-Bzl)-NCA

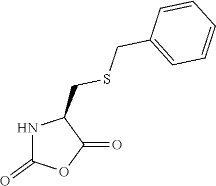 [SMILES: O=C1N[C@@H](CSCc2ccccc2)C(=O)O1]

Recrystallized in an AcOEt/hexane mixture

Yield 91%

White solid, mp 100-102° C., [α]20_{D}=−60 (c=1.00, CH₂Cl₂).

### Example 29: Preparation of N-Me(L)Ala-NCA

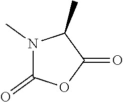

Recrystallized in an Ether/hexane mixture

Yield 87%

White solid, mp 131-132° C., [α]20_{D}=+10 (c=1.00, CH2Cl2), ¹H NMR (600 MHz, DMSO-d₆) δ 4.42-4.37 (dd, J=7.13, 14.06 Hz, 1H), 2.84 (s, 3H), 1.39 (d, J=7.13 Hz); ¹³C NMR (100 MHz, DMSO-d₆): δ 171.2, 152.1, 57.2, 28.4, 14.5.

### Example 30: Preparation of N-Benzyl-Gly-NCA

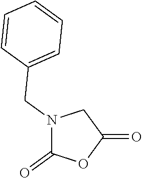 [SMILES: O=C1CN(Cc2ccccc2)C(=O)O1]

Recrystallized in an Ether/hexane mixture

Yield 82%

White solid, mp 152-153° C., ¹H NMR (600 MHz, DMSO-d₆) δ 7.37 (m, 5H), 4.50 (s, 2H), 4.17 (s, 2H); ¹³C NMR (100 MHz, DMSO-d₆): δ 167.5, 153.1, 135.7, 129.1, 128.2, 128.2, 50.0, 47.1.

### Example 31: Preparation of (L)Pro-NCA

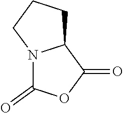

Recrystallized in an AcOEt/hexane mixture

Yield 85%

White solid, mp 45-47° C., [α]²⁰_{D}=−99 (c=1.00, CH₂Cl₂).

### Example 32: Preparation of (D)Asp(OBzl)-NCA

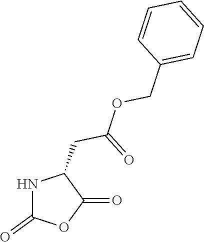 [SMILES: O=C(C[C@H]1NC(=O)OC1=O)OCc1ccccc1]

Recrystallized in an AcOEt/hexane mixture

Yield 85%

White solid, mp 123-124° C., [α]19_{D}=+42.3 (c=1.00, CH2 Cl2) ¹H NMR (600 MHz, DMSO-d₆) δ 8.99 (s, 1H), 7.41-7.34 (m, 5H), 5.13 (s, 2H), 4.71-4.69 (t, J=4.57 Hz, 1H), 3.11-3.05 (dd, J=4.86, 17.87 Hz, 1H), 2.93-2.87 (dd, J=4.86, 17.87 Hz, 1H); ¹³C NMR (100 MHz, DMSO-d₆): δ 171.4, 169.7, 152.6, 136.0, 128.9, 128.6, 128.5, 66.7, 54.0, 35.1.

### Example 33: Preparation of Aib-NCA

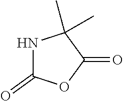

Recrystallized in an AcOEt/hexane mixture

Yield 82%

White solid, mp 72-73° C., ¹H NMR (400 MHz, DMSO-d₆): δ 9.07 (s, 1H), 1.41 (s, 6H); ¹³C NMR (100 MHz, DMSO-d₆): 175.0, 150.8, 59.6, 25.0.

### Example 34: Preparation of (L)Asn(Xan)-NCA

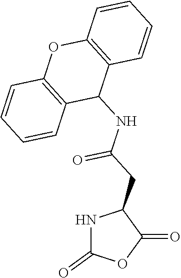 [SMILES: O=C(C[C@@H]1NC(=O)OC1=O)NC1c2ccccc2Oc2ccccc21]

Recrystallized in an AcOEt/hexane mixture

Yield 89%

White solid, mp 145-146° C., [α]20_{D}=−55.6 (c=1.00, dioxane), ¹H NMR (400 MHz, DMSO-d₆): δ 9.08-9.06 (d, J=8.80 Hz, 1H), 8.97 (s, 1H), 7.39-7.33 (m, 4H), 7.18-7.14 (m, 4H), 6.30-6.28 (d, J=8.80 Hz, 1H), 4.66-4.64 (t, J=4.46 Hz, 1H), 2.86-2.81 (dd, J=4.33, 16.82 Hz, ^{2H}) 2.74-2.69 (dd, J=4.33, 16.82 Hz, 2H); 13C NMR (100 MHz, DMSO-d₆): δ 172.1, 167.9, 152.9, 150.9, 129.6, 124.0, 121.9, 121.8, 116.6, 54.3, 43.1, 36.2.

1.-15. (canceled)

16. A method for preparing of a NCA compound, comprising:
a step of contacting a N-protected α-amino-acid compound with propane-phosphonic acid anhydride, in an organic solvent, to obtain said compound NCA.

17. The method of preparation according to claim 16, wherein said compound NCA is of Formula 1-A, prepared from a N-protected α-amino-acid compound of Formula 2-A, according to the following reaction scheme: 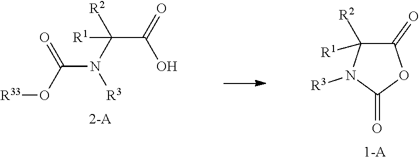
Formula 1-A wherein:
R¹ and R² independently represent:
a hydrogen atom,
a group chosen from:
a linear or branched C₁ to C₂₀ alkyl,
a linear or branched C₂ to C₂₀ alkenyl,
C₃ to C₁₀ cycloalkyl,
C₃ to C₁₀ heterocycloalkyl, wherein the heteroatom is selected from N, O, and S, aryle,
C₁ to C₂₀ alkyl-aryl, in particular benzyl,
heteroaryl, wherein the heteroatom is selected from N, O, and S, in particular 3-methylindole,
C₁ to C₂₀ alkyl-heteroaryl, wherein the heteroatom is selected from N, O, and S,
a halogen, in particular a fluorine atom,
said alkyl, alkyl-aryl or alkyl-heteroaryl may be substituted on at least one carbon of the alkyl radical by one or more groups selected from:
O—R⁴, wherein R⁴ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
O—C(O)—R⁵, wherein R⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
C(O)—R⁶, wherein R⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
C(O)—O—R⁷, wherein R⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
NR⁸R⁹, wherein R⁸ and R⁹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
(NH)CNHR¹⁰, wherein R10 is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
NR¹¹C(O)R¹², wherein R¹¹ and R¹² are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R¹² being in particular a protective group such as Boc, Cbz, Alloc or Fmoc,
C(O)—NR¹³R¹⁴, wherein R¹³ and R¹⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
S—R¹⁵, wherein R¹⁵ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
a halogen, in particular selected from F, Cl, Br and I,
said aryl, alkyl-aryl, heteroaryl and alkyl-heteroaryl may be substituted on the aromatic or heteroaromatic ring by one or more groups selected from:
a linear or branched C₁ to C₂₀ alkyl,
C₃ to C₁₀ cycloalkyl,
C₃ to C₁₀ heterocycloalkyl,
O—R¹⁶, wherein R¹⁶ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular selected from tBDMS, t-butyl, benzyl, trityl and xanthyl,
O—C(O)—R¹⁷, wherein R¹⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
C(O)—R¹⁸, wherein R¹⁸ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
C(O)—O—R¹⁹, wherein R¹⁹ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
NR²⁰R²¹, wherein R²⁰ and R²¹ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
(NH)CNHR²², wherein R²² is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular NO₂, Pbf, Pmc, Mtr or Boc,
NR²³C(O)R²⁴, wherein R²³ and R²⁴ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, linear or branched C₁ to C₂₀ alkyl-aryl, heteroaryl, linear or branched C₁ to C₂₀ alkyl-heteroaryl, linear or branched C₁ to C₂₀ O-alkyl, linear or branched C₁ to C₂₀ O-alkyl-aryl, the radical —C(O)R²⁴ being in particular a protective group such as Boc, Cbz, Alloc or Fmoc,
C(O)—NR²⁵R²⁶, wherein R²⁵ and R²⁶ are independently selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl,
S—R²⁷, wherein R²⁷ is selected from H, linear or branched C₁ to C₂₀ alkyl, linear or branched C₁ to C₂₀ heteroalkyl, C₃ to C₁₀ cycloalkyl, C₃ to C₁₀ heterocycloalkyl, aryl, C₁ to C₂₀ alkyl-aryl, heteroaryl and C₁ to C₂₀ alkyl-heteroaryl and a protective group, in particular trityl or acetamidomethyl (Acm),
a halogen, in particular selected from F, Cl, Br and I,
R¹ and R² may form a cycle,
one of the groups R¹ or R² is in particular a hydrogen atom,
R³ represents:
a hydrogen atom,
a linear or branched C₁ to C₂₀ alkyl group,
R³ is in particular a hydrogen atom,
said group R³ may form a cycle with R¹ or R²,
said compound of Formula 1 may be in the form of a solvate or a hydrate,
said groups NR⁸R⁹, (NH)CNHR¹⁰, NR²⁰R²¹ and (NH)CNHR²², heteroaryl, alkyl-heteroaryl and/or heterocycloalkyl may be in a salified form, and
Formula 2-A wherein R¹, R² and R³ are as defined above for Formula 1-A, and wherein R³³ represents a linear or branched C₁ to C₂₀ alkyl group, in particular a tert-butyl,
when the compound of Formula 1-A, or the compound of Formula 2-A, comprises a carbon atom, said carbon atom may be ¹³C,
when the compound of Formula 1-A, or the compound of Formula 2-A, comprises a fluorine atom, said fluorine atom may be ¹⁸F,
when the compound of Formula 1-A, or the compound of Formula 2-A, comprises a hydrogen atom, said hydrogen atom may be deuterium,
the asymmetric centers of said compound of Formula 1-A, and said compound of Formula 2-A, are of R or S configuration, or a mixture thereof.

18. The method according to claim 16, wherein the compound of Formula 1-A and the compound of Formula 2-A are such that:
R¹ and R² independently represent:
a hydrogen atom,
a group chosen from:
a linear or branched C₁ to C₁₀ alkyl,
3-methylindole,
said alkyl may be substituted by one or more groups selected from:
O—R⁴, wherein R⁴ is a t-butyl group,
(NH)CNHR¹⁰, wherein R¹⁰ is selected from a protective group, in particular NO₂, Pbf, Pmc, Mtr or Boc,
NR¹¹C(O)R¹², wherein R¹¹ is H, and the radical —C(O) R¹² is a protective group such as Boc, Cbz, Alloc or Fmoc,
S—R¹⁵, wherein R¹⁵ is trityl or acetamidomethyl (Acm),
R³ represents:
a hydrogen atom,
a linear or branched C₁ to C₂₀ alkyl group,
said group R³ may form a cycle with R¹ or R²,
R³³ represents a tert-butyl group,
said compound of Formula 1-A may be in the form of a solvate or hydrate,
said groups NR⁸R⁹, (NH)CNHR¹⁰, NR²⁰R²¹ and (NH) CNHR²² and/or 3-methylindole, of the compound of Formula 1-A may be in a salified form,
the asymmetric centers of said compound of Formula 1-A, and
said compound of Formula 2-A, are of R or S configuration, or a mixture thereof.

19. The method according to claim 16, wherein the α-amine acid compound is selected from:
alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, 1,2,3,4-tetrahydroisoquinoline-3-carboxylic acid (Tic), 2-amino-2-methylpropanoic acid (Aib), and norleucine (Nle),
said α-amino-acid compound being N-protected on the α amine function by a linear or branched C₁ to C₂₀—C(O)—O-alkyl substituent, in particular by a -tert-butyloxycarbonyl group,
said amino acids—being optionally protected, on the side chain, on the carboxylic acid functions, amine functions, thiol functions, guanidine functions, amide functions and/or alcohol functions, by a protective group, in particular selected from tert-butyloxycarbonyl (Boc), benzyloxycarbonyl (Cbz) fluorenylmethyloxycarbonyl (Fmoc), alloc, tert-butyloxy (OtBu), formyl (For), 2,2,4,6, 7-pentamethylhydrobenzofuran-5-sulfonyl (Pbf), 2,2,5,7,8-pentamethylchroman-6-sulfonyl (Pmc), 4-methoxy-2,3,6-trimethylbenzenesulfonyl (Mtr), trityl (Trt), trifluoroacetyl, acetamidomethyl (Acm), and xanthyl (Xan).

20. The method according to claim 16, wherein the compound NCA is selected from the following structures:

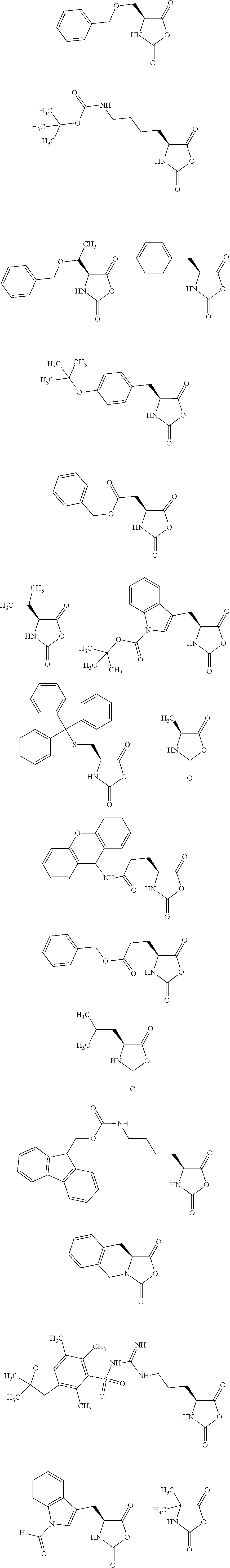

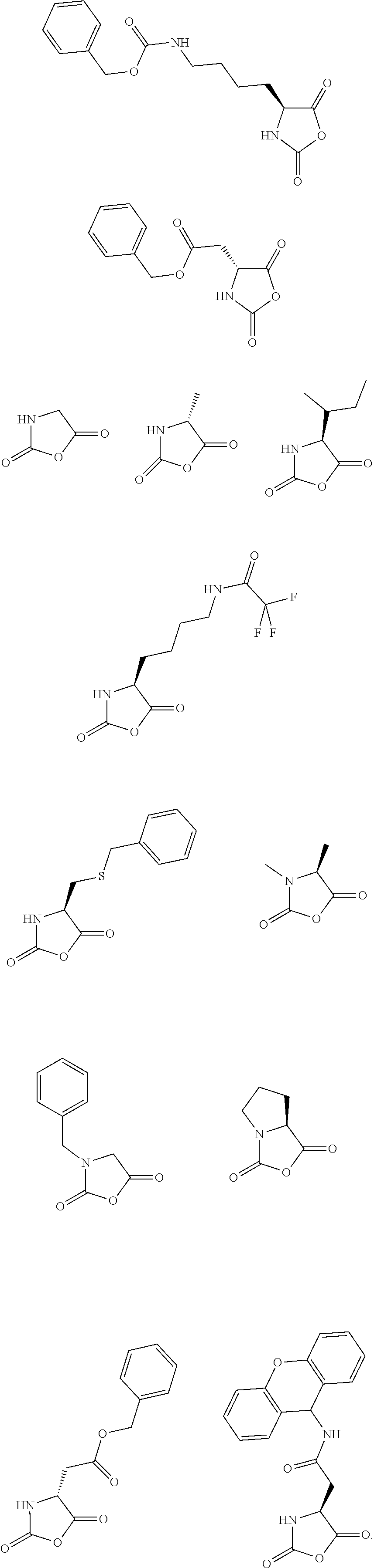

21. The method according to claim 16, wherein the step of contacting a N-protected α-amino-acid compound with propane-phosphonic acid anhydride is in the presence of an organic base, at room temperature.

22. The method according to claim 21, wherein:
said organic base being selected in particular from triethylamine, 1,8-diazabicyclo [5.4.0] undec-7-ene, diisopropylethylamine, N-dimethylaminopyridine, N-methylmorpholine or pyridine.

23. The method according to claim 16, wherein the step of contacting a N-protected α-amino-acid compound with propane-phosphonic acid anhydride is in the absence of an organic base, at a temperature between 40 and 80° C.

24. The method according to claim 16, further comprising:
after obtaining the NCA, a step of purification by at least one aqueous wash.

25. The method according to claim 16, further comprising:
a step of purification of the NCA compound,
wherein said α-amino-acid compound is N-protected on the α amine function by a linear or branched C₁ to C₂₀ C(O)—O-alkyl substituent.

26. The method according to claim 16, wherein:
said organic solvent is selected from ethyl acetate or dimethylformamide.

27. The method according to claim 16, wherein:
the preparation of the NCA compound is done in the presence of an organic base in an amount of 0.25 to 3 molar equivalents relative to the N-protected α-amino-acid compound.

28. The method according to claim 16, wherein:
propane-phosphonic acid anhydride is used in an amount of 1 to 4 molar equivalents relative to the N-protected α-amino-acid compound.

29. The method according to claim 16, wherein:
the step of purification of the NCA compound, is a recrystallization step.

30. The method according to claim 16, wherein said α-amino-acid compound is N-protected on the α amine function by a -tert-butyloxycarbonyl group, said method being optionally implemented in continuous flow.

31. An NCA compound, of the following structure:

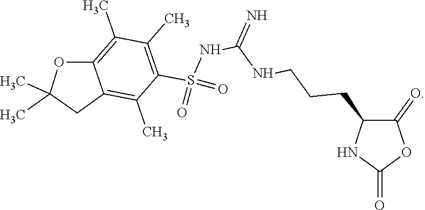 [SMILES: Cc1c(C)c(S(=O)(=O)NC(=N)NCCC[C@@H]2NC(=O)OC2=O)c(C)c2c1OC(C)(C)C2]

32. A solution comprising an NCA compound according to claim 31, said solution being devoid of phosgene decomposition products, diphosgene decomposition products and triphosgene decomposition products, in particular devoid of hydrochloric acid.

33. A solution comprising an NCA compound prepared according to claim 16, said solution being devoid of phosgene decomposition products, diphosgene decomposition products and triphosgene decomposition products, in particular devoid of hydrochloric acid.
